SEQUENCE LISTING (54 sequences)

SEQ ID NO: 1 - protein, 8 aa, Artificial Sequence
  Description of Artificial Sequence Synthetic peptide
GFNIKDSF

SEQ ID NO: 2 - protein, 8 aa, Artificial Sequence
  Description of Artificial Sequence Synthetic peptide
IDPEDDET

SEQ ID NO: 3 - protein, 14 aa, Artificial Sequence
  Description of Artificial Sequence Synthetic peptide
ASPIYGSREAWFAY

SEQ ID NO: 4 - protein, 6 aa, Artificial Sequence
  Description of Artificial Sequence Synthetic peptide
EDIYSN

SEQ ID NO: 5 - protein, 3 aa, Artificial Sequence
  Description of Artificial Sequence Synthetic peptide
DAS

SEQ ID NO: 6 - protein, 9 aa, Artificial Sequence
  Description of Artificial Sequence Synthetic peptide
QQHHDYPLT

SEQ ID NO: 7 - protein, 6 aa, Artificial Sequence
  Description of Artificial Sequence Synthetic peptide
EDIYNG

SEQ ID NO: 8 - protein, 3 aa, Artificial Sequence
  Description of Artificial Sequence Synthetic peptide
NAN

SEQ ID NO: 9 - protein, 9 aa, Artificial Sequence
  Description of Artificial Sequence Synthetic peptide
QQYYNYPYT

SEQ ID NO: 10 - protein, 6 aa, Artificial Sequence
  Description of Artificial Sequence Synthetic peptide
  MOD_RES (5)..(5): A polar amino acid
  MOD_RES (6)..(6): S, T, N, Q, C, G or P
EDIYXX

SEQ ID NO: 11 - protein, 3 aa, Artificial Sequence
  Description of Artificial Sequence Synthetic peptide
  MOD_RES (1)..(1): D, E, S, T, N or Q
  MOD_RES (3)..(3): A polar amino acid
XAX

SEQ ID NO: 12 - protein, 9 aa, Artificial Sequence
  Description of Artificial Sequence Synthetic peptide
  MOD_RES (3)..(3): R, H, K, A, V, I, L, M, F, Y or W
  MOD_RES (4)..(4): R, H, K, A, V, I, L, M, F, Y or W
  MOD_RES (5)..(5): D, E, S, T, N or Q
  MOD_RES (8)..(8): A, V, I, L, M, F, Y or W
QQXXXYPXT

SEQ ID NO: 13 - protein, 121 aa, Artificial Sequence
  Description of Artificial Sequence Synthetic polypeptide
EVQLQQSGAELVRPGASVRLSCTTSGFNIKDSFIHWVKQRTEQGLEWIGRIDPEDDETKYAPKFQGKATITADTSSNTAYLQLSSLTSEDTAVYYCASPIYGSREAWFAYWGQGTLVTVSA

SEQ ID NO: 14 - protein, 107 aa, Artificial Sequence
  Description of Artificial Sequence Synthetic polypeptide
DIQMTQSPASLSASLGETVTIECLASEDIYSNLAWYQQKPGKSPQLLIYDASSLQDGVPSRFSGSESGTQYSLEINSLQSEDAATYFCQQHHDYPLTFGSGTKLEIK

SEQ ID NO: 15 - protein, 107 aa, Artificial Sequence
  Description of Artificial Sequence Synthetic polypeptide
DIQMTQSPASLSASLGETVTIECRASEDIYNGLAWYQQKPGKSPQLLIYNANSLHTGVPSRFSGSGSGTQYSLKINSLQSEDVASYFCQQYYNYPYTFGAGTKLELK

SEQ ID NO: 16 - DNA, 363 bp, Artificial Sequence
  Description of Artificial Sequence Synthetic polynucleotide
gaggttcagctgcagcagtctggggcagagcttgtgaggccaggggcctcagtcaggttgtcctgcacaacttctggcttcaacattaaagactcctttattcactgggtgaagcagaggactgaacagggcctggagtggattggaaggattgatcctgaggatgatgaaactaaatatgccccgaaattccagggcaaggccactataacagcagacacatcctccaacacagcctacctgcagctcagcagcctgacatctgaggacactgccgtctattactgtgctagccccatctacggtagtagagaggcctggtttgcttactggggccaagggactctggtcactgtctctgca

SEQ ID NO: 17 - DNA, 321 bp, Artificial Sequence
  Description of Artificial Sequence Synthetic polynucleotide
gacatccagatgacacagtctccagcttccctgtctgcatctctgggagaaactgtcaccatcgaatgtctagcaagtgaagacatttacagtaatttagcgtggtatcagcagaagccagggaaatctcctcagctcctgatctatgatgcaagtagcttgcaagatggggtcccatcacggttcagtggcagtgaatctggcacacagtattctctcgagatcaacagcctgcaatctgaagatgccgcgacttatttctgtcaacagcatcatgattatcctctcacgttcggttctgggaccaagctggagatcaaa

SEQ ID NO: 18 - DNA, 363 bp, Artificial Sequence; identical to SEQ ID NO: 16
  Description of Artificial Sequence Synthetic polynucleotide

SEQ ID NO: 19 - DNA, 321 bp, Artificial Sequence
  Description of Artificial Sequence Synthetic polynucleotide
gacatccagatgacacagtctccagcttccctgtctgcatctctgggagaaactgtcaccatcgaatgtcgagcaagtgaggacatttacaatggtttagcatggtatcagcagaagccagggaaatctcctcagctcctgatctataatgcaaatagcttgcatactggggtcccatcacggttcagtggcagtggatctggtacacagtattctctcaagataaacagcctgcagtctgaagatgtcgcaagttatttctgtcaacagtattacaattatccgtacacgtttggagctgggaccaagctggaactgaaa

SEQ ID NO: 20 - protein, 107 aa, Artificial Sequence
  Description of Artificial Sequence Synthetic polypeptide
DVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIK

SEQ ID NO: 21 - protein, 115 aa, Artificial Sequence
  Description of Artificial Sequence Synthetic polypeptide
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSS

SEQ ID NO: 22 - protein, 111 aa, Artificial Sequence
  Description of Artificial Sequence Synthetic polypeptide
DIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELK

SEQ ID NO: 23 - protein, 118 aa, Artificial Sequence
  Description of Artificial Sequence Synthetic polypeptide
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSS

SEQ ID NO: 24 - protein, 41 aa, Artificial Sequence
  Description of Artificial Sequence Synthetic polypeptide
RSKRSRLLHSDYMNMTPRRPGPTRKHQYPYAPPRDFAAYRS

SEQ ID NO: 25 - protein, 41 aa, Artificial Sequence
  Description of Artificial Sequence Synthetic polypeptide
RSKRSRGGHSDYMNMTPRRPGPTRKHYQPYAPPRDFAAYRS

SEQ ID NO: 26 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 27 - protein, 42 aa, Artificial Sequence
  Description of Artificial Sequence Synthetic polypeptide
ALYLLRRDQRLPPDAHKPPGGGSFRTPIQEEQADAHSTLAKI

SEQ ID NO: 28 - protein, 21 aa, Artificial Sequence
  Description of Artificial Sequence Synthetic peptide
LCYLLDGILFIYGVILTALFL

SEQ ID NO: 29 - protein, 27 aa, Artificial Sequence
  Description of Artificial Sequence Synthetic peptide
FWVLVVVGGVLACYSLLVTVAFIIFWV

SEQ ID NO: 30 - protein, 28 aa, Artificial Sequence
  Description of Artificial Sequence Synthetic peptide
MFWVLVVVGGVLACYSLLVTVAFIIFWV

SEQ ID NO: 31 - protein, 22 aa, Artificial Sequence
  Description of Artificial Sequence Synthetic peptide
MALIVLGGVAGLLLFIGLGIFF

SEQ ID NO: 32 - protein, 21 aa, Artificial Sequence
  Description of Artificial Sequence Synthetic peptide
IYIWAPLAGTCGVLLLSLVIT

SEQ ID NO: 33 - protein, 23 aa, Artificial Sequence
  Description of Artificial Sequence Synthetic peptide
IYIWAPLAGTCGVLLLSLVITLY

SEQ ID NO: 34 - protein, 24 aa, Artificial Sequence
  Description of Artificial Sequence Synthetic peptide
IYIWAPLAGTCGVLLLSLVITLYC

SEQ ID NO: 35 - protein, 24 aa, Artificial Sequence
  Description of Artificial Sequence Synthetic peptide
IISFFLALTSTALLFLLFFLTLRF

SEQ ID NO: 36 - protein, 3 aa, Artificial Sequence
  Description of Artificial Sequence Synthetic peptide
AAA

SEQ ID NO: 37 - protein, 10 aa, Artificial Sequence
  Description of Artificial Sequence Synthetic peptide
GGGSSGGGSG

SEQ ID NO: 38 - protein, 12 aa, Artificial Sequence
  Description of Artificial Sequence Synthetic peptide
ESKYGPPCPPCP

SEQ ID NO: 39 - protein, 12 aa, Artificial Sequence
  Description of Artificial Sequence Synthetic peptide
ESKYGPPCPSCP

SEQ ID NO: 40 - protein, 22 aa, Artificial Sequence
  Description of Artificial Sequence Synthetic peptide
ESKYGPPCPPCPGGGSSGGGSG

SEQ ID NO: 41 - protein, 39 aa, Artificial Sequence
  Description of Artificial Sequence Synthetic polypeptide
IEVMYPPPYLDNEKSNGTIIHVKGKHLCPSPLFPGPSKP

SEQ ID NO: 42 - protein, 48 aa, Artificial Sequence
  Description of Artificial Sequence Synthetic polypeptide
AKPTTTPAPRPPTPAPTIASQPLSLRPEACRPAAGGAVHTRGLDFACD

SEQ ID NO: 43 - protein, 45 aa, Artificial Sequence
  Description of Artificial Sequence Synthetic polypeptide
TTTPAPRPPTPAPTIASQPLSLRPEACRPAAGGAVHTRGLDFACD

SEQ ID NO: 44 - protein, 129 aa, Artificial Sequence
  Description of Artificial Sequence Synthetic polypeptide
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 229 aa, Artificial Sequence
  Description of Artificial Sequence Synthetic polypeptide
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 229 aa, Artificial Sequence
  Description of Artificial Sequence Synthetic polypeptide
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 107 aa, Artificial Sequence
  Description of Artificial Sequence Synthetic polypeptide
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 650 aa, Artificial Sequence
  Description of Artificial Sequence Synthetic polypeptide
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 657 aa, Artificial Sequence
  Description of Artificial Sequence Synthetic polypeptide
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 22 aa, Artificial Sequence
  Description of Artificial Sequence Synthetic peptide
MLLLVTSLLLCELPHPAFLLIP

SEQ ID NO: 52 - protein, 364 aa, Artificial Sequence
  Description of Artificial Sequence Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMGMCSFRA

SEQ ID NO: 53 - protein, 323 aa, Artificial Sequence
  Description of Artificial Sequence Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 54 - protein, 24 aa, Artificial Sequence
  Description of Artificial Sequence Synthetic peptide
LEGGGEGRGSLLTCGDVEENPGPR

(19) United States
(12) Patent Application Publication
(10) Pub. No.: US 2021/0277149 A1
(43) Pub. Date: Sep. 9, 2021

(54) ANTI-IDIOTYPE ANTIBODIES AND METHODS OF USING THE SAME

(71) Applicant: Mustang Bio, Inc., Worchester, MA (US)

(72) Inventors: Ekta PATEL, Lexington, MA (US); Junxia WANG, Wellesley, MA (US); Ajit KAMATH, Natick, MA (US); Nathan K GUMLAW, Holland, MA (US)

(73) Assignee: Mustang Bio, Inc., Worchester, MA (US)

(21) Appl. No.: 17/191,694

(22) Filed: Mar. 3, 2021

Related U.S. Application Data
(60) Provisional application No. 62/986,405, filed on Mar. 6, 2020.

(51) Int. Cl. C07K 16/42; C12N 5/0783; G01N 33/569; G01N 33/68

(52) U.S. Cl. CPC C07K 16/4258; C12N 5/0636; C12N 5/0646; C12N 2501/50; G01N 33/686; C07K 2317/565; G01N 33/56972

(57) ABSTRACT

The present disclosure relates generally to antibodies and binding fragments thereof that bind to anti-CD123 antibodies, chimeric antigen receptors (CARs), or antibody binding fragments. In particular, the disclosed anti-idiotype antibodies and fragments bind to anti-CD123 antibodies, CARs, or fragments thereof and comprise novel complementary determining regions (CDRs). Finally, the present disclosure relates to methods of using the disclosed antibodies and fragments thereof to expand and/or activate CD123-CAR-expressing immune cells, detecting or quantifying CD123-CARs, and isolating CD123-CAR-expressing immune cells.

### CROSS-REFERENCE TO RELATED APPLICATIONS

This application claims priority from U.S. Provisional Patent Application No. 62/986,405, filed Mar. 6, 2020. The contents of this application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Apr. 21, 2021, is named 118180-0401_SL.txt and is 47,377 bytes in size.

### FIELD OF INVENTION

The present disclosure relates generally to antibodies and binding fragments thereof that bind to anti-CD123 antibodies, chimeric antigen receptors (CARs), or antibody binding fragments. In particular, the disclosed anti-idiotype antibodies and fragments bind to anti-CD123 antibodies, CARs, or fragments thereof and comprise novel complementary determining regions (CDRs). Finally, the present disclosure relates to methods of using the disclosed antibodies and fragments thereof to expand and/or activate CD123-CAR-expressing immune cells, detecting or quantifying CD123-CARs, and isolating CD123-CAR-expressing immune cells.

### BACKGROUND

The following discussion is merely provided to aid the reader in understanding the disclosure and is not admitted to describe or constitute prior art thereto.

CD123 (i.e., interleukin 3 receptor alpha chain; IL-3Rα) belongs to the type I cytokine receptor family and is a heterodimer with a unique alpha chain paired with the common beta (beta c or CD131) subunit. CD123 is expressed on various malignancies including acute and chronic myeloid leukemia, hairy cell leukemia, B-cell lineage acute lymphoblastic leukemia, and blastic plasmacytoid dendritic cell neoplasms. Additionally, CD123 is not typically expressed on normal hematopoietic stem cells, thus making CD123 an ideal immunotherapeutic target.

Immunotherapies like antibodies and chimeric antigen receptor (CAR)-expressing immune cells (e.g., T cells or natural killer cells) hold great potential for treating various types of cancer using target-specific mechanisms. However, isolating and quantifying such antibodies or CAR-expressing cells can be laborious and difficult, and expansion and activation of CAR-expressing cells can be challenging.

The present disclosure provides anti-idiotype antibodies and fragments that bind to anti-CD123 antibodies, CARs, or fragments thereof, and which are useful for various different manufacturing, quality control, and therapeutic applications.

### SUMMARY

Described herein are novel anti-idiotype antibodies that bind to anti-CD123 antibodies or fragments thereof and methods of using the same.

In one aspect, the disclosure relates to An antibody or antigen-binding fragment comprising a heavy chain variable (V_{H}) region and a light chain variable (V_{L}) region, the V_{H} and V_{L} regions comprising a heavy chain complementarity determining region 1 (CDRH1) comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a light chain complementarity determining region 1 (CDRL1) comprising EDIYX₁X₂ (SEQ ID NO: 10), a CDRL2 comprising X₃AX₄ (SEQ ID NO: 11), and a CDRL3 comprising QQX₅X₆X₇YPX₈T (SEQ ID NO: 12), wherein X₁ is a polar amino acid; X₂ is selected from the group consisting of serine (S), threonine (T), asparagine (N), glutamine (Q), cysteine (C), glycine (G), and proline (P); X₃ is selected from the group consisting of aspartic acid (D), glutamic acid (E), serine (S), threonine (T), asparagine (N), and glutamine (Q); X₄ is a polar amino acid; X₅ and X₆ are selected from the group consisting of arginine (R), histidine (H), lysine (K), alanine (A), valine (V), isoleucine (I), leucine (L), methionine (M), phenylalanine (F), tyrosine (Y), and tryptophan (W); X₇ is selected from the group consisting of aspartic acid (D), glutamic acid (E), serine (S), threonine (T), asparagine (N), and glutamine (Q); and X₈ is selected from the group consisting of alanine (A), valine (V), isoleucine (I), leucine (L), methionine (M), phenylalanine (F), tyrosine (Y), and tryptophan (W).

In some embodiments, X₁ is serine (S) or asparagine (N). In some embodiments, X₂ is asparagine (N) or glycine (G). In some embodiments, X₃ is aspartic acid (D) or asparagine (N). In some embodiments, X₄ is serine (S) or asparagine (N). In some embodiments, X₅ and X₆ are histidine (H) or tyrosine (Y). In some embodiments, X₇ is aspartic acid (D) or asparagine (N). In some embodiments, X₈ is leucine (L) or tyrosine (Y). In some embodiments, X₁ is serine (S) or asparagine (N); X₂ is asparagine (N) or glycine (G); X₃ is aspartic acid (D) or asparagine (N); wherein X₄ is serine (S) or asparagine (N); X₅ and X₆ are histidine (H) or tyrosine (Y); X₇ is aspartic acid (D) or asparagine (N); and X₈ is leucine (L) or tyrosine (Y).

In some embodiments, the antibody or antigen-binding fragment may comprise: a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYSN (SEQ ID NO: 4), a CDRL2 comprising DAS (SEQ ID NO: 5), and a CDRL3 comprising QQHHDYPLT (SEQ ID NO: 6); or a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYNG (SEQ ID NO: 7), a CDRL2 comprising NAN (SEQ ID NO: 8), and a CDRL3 comprising QQYYNYPYT (SEQ ID NO: 9).

In some embodiments, the antibody or antigen-binding fragment may comprise V_{H} region comprising EVQLQQSGAELVRPGASVRLSCTTSGFNIKDSFIHWVKQRTEQGLEWIGRI DPEDDETKYAPKFQGKATITADTSSNTAYLQLSSLTSEDTAVYYCASPIYGSREAWFAY WGQGTLVTVSA (SEQ ID NO: 13) and a V_{L}, region comprising DIQMTQSPASLSASLGETVTIECLASEDIYSNLAWYQQKPGKSPQLLIYDASSLQDGVPS RFSGSESGTQYSLEINSLQSEDAATYFCQQHHDYPLTFGSGTKLEIK (SEQ ID NO: 14); or a V_{H} region comprising EVQLQQSGAELVRPGASVRLSCTTSGFNIKDSFIHWVK QRTEQGLEWIGRIDPEDDETKYAPKFQGKATITADTSSNTAYLQLSSLTSEDTAVYYCAS PIYGSREAWFAYWGQGTLVTVSA (SEQ ID NO: 13) and a V_{L}, region comprising DIQMTQSPASLSASLGETVTIECRASEDIYNGLAWYQQKPGKSPQLLIYNANSLHTGVPS RFSGSGSGTQYSLKINSLQSEDVASYFCQQYYNYPYTFGAGTKLELK (SEQ ID NO: 15).

In some embodiments, the antibody or antigen-binding fragment specifically binds to an idiotype on an anti-CD123 antibody or an anti-CD123 antigen-binding fragment. In some embodiments, the anti-CD123 antibody or anti-CD123 antigen-binding fragment comprises the V_{L} domain of DVQITQSPSYLAASPGETITINCRASKSISKDLAWYQEKPGKTNKLLIYSGSTLQ SGIPSRFSGSGSGTDFTLTISSLEPEDFAMYYCQQHNKYPYTFGGGTKLEIK (SEQ ID NO: 20) and/or the V_{H} domain of QVQLQQPGAELVRPGASVKLSCKASGYTFTSYWM NWVKQRPDQGLEWIGRIDPYDSETHYNQKFKDKAILTVDKSSSTAYMQLSSLTSEDSAV YYCARGNWDDYWGQGTTLTVSS (SEQ ID NO: 21). In some embodiments, the anti-CD123 antigen-binding fragment is a scFv. In some embodiments, the anti-CD123 antigen-binding fragment is incorporated into a chimeric antigen receptor (CAR).

In those embodiments in which the anti-idiotype antibody or fragment binds a CAR, the CAR may comprise an IgG hinge or a modified IgG hinge, a transmembrane domain, a co-stimulatory signaling domain, and a T cell receptor zeta chain signaling domain. In some embodiments, the co-stimulatory signaling domain is selected from the group consisting of: a CD27 co-stimulatory signaling domain, a CD28 co-stimulatory signaling domain, a 4-1BB co-stimulatory signaling domain, and an OX40 co-stimulatory signaling domain. In some embodiments, the transmembrane domain comprises a transmembrane portion of CD28, CD4, CD8, 4-1BB, CD27, ICOS, OX40, HVEM, or CD30. In some embodiments, the CD123-CAR comprises a V_{L} domain comprising SEQ ID NO: 20 and V_{H} domain comprising SEQ ID NO: 21, a CD28 transmembrane domain, a co-stimulatory domain comprising SEQ ID NO: 24 or SEQ ID NO: 25, and a CD3ζ domain comprising SEQ ID NO: 48. In some embodiments, the CD123-CAR comprises SEQ ID NO: 49. In some embodiments, the CD123-CAR comprises a V_{L} domain comprising SEQ ID NO: 22 and V_{H} domain comprising SEQ ID NO: 23, a CD28 transmembrane domain, a co-stimulatory domain comprising SEQ ID NO: 24 or SEQ ID NO: 25, and a CD3ζ domain comprising SEQ ID NO: 48. In some embodiments, the CD123-CAR comprises SEQ ID NO: 50.

In another aspect, the present disclosure provides nucleotide sequences encoding an antibody or antigen-binding fragment comprising:

GAGGTTCAGCTGCAGCAGTCTGGGGCAGAGCTTGTGAGGCCAGGGGCCTCAGTCAG GTTGTCCTGCACAACTTCTGGCTTCAACATTAAAGACTCCTTTATTCACTGGGTGAAG CAGAGGACTGAACAGGGCCTGGAGTGGATTGGAAGGATTGATCCTGAGGATGATGA AACTAAATATGCCCCGAAATTCCAGGGCAAGGCCACTATAACAGCAGACACATCCT CCAACACAGCCTACCTGCAGCTCAGCAGCCTGACATCTGAGGACACTGCCGTCTATT ACTGTGCTAGCCCCATCTACGGTAGTAGAGAGGCCTGGTTTGCTTACTGGGGCCAAG GGACTCTGGTCACTGTCTCTGCA (SEQ ID NO: 16) and GACATCCAGATGACACAGTCTCCAGCTTCCCTGTCTGCATCTCTGGGAGAAACTGTC ACCATCGAATGTCTAGCAAGTGAAGACATTTACAGTAATTTAGCGTGGTATCAGCAG AAGCCAGGGAAATCTCCTCAGCTCCTGATCTATGATGCAAGTAGCTTGCAAGATGGG GTCCCATCACGGTTCAGTGGCAGTGAATCTGGCACACAGTATTCTCTCGAGATCAAC AGCCTGCAATCTGAAGATGCCGCGACTTATTTCTGTCAACAGCATCATGATTATCCT CTCACGTTCGGTTCTGGGACCAAGCTGGAGATCAAA (SEQ ID NO: 17); or
GAGGTTCAGCTGCAGCAGTCTGGGGCAGAGCTTGTGAGGCCAGGGGCCTCAGTCAG GTTGTCCTGCACAACTTCTGGCTTCAACATTAAAGACTCCTTTATTCACTGGGTGAAG CAGAGGACTGAACAGGGCCTGGAGTGGATTGGAAGGATTGATCCTGAGGATGATGA AACTAAATATGCCCCGAAATTCCAGGGCAAGGCCACTATAACAGCAGACACATCCT CCAACACAGCCTACCTGCAGCTCAGCAGCCTGACATCTGAGGACACTGCCGTCTATT ACTGTGCTAGCCCCATCTACGGTAGTAGAGAGGCCTGGTTTGCTTACTGGGGCCAAG GGACTCTGGTCACTGTCTCTGCA (SEQ ID NO: 18) and GACATCCAGATGACACAGTCTCCAGCTTCCCTGTCTGCATCTCTGGGAGAAACTGTC ACCATCGAATGTCGAGCAAGTGAGGACATTTACAATGGTTTAGCATGGTATCAGCA GAAGCCAGGGAAATCTCCTCAGCTCCTGATCTATAATGCAAATAGCTTGCATACTGG GGTCCCATCACGGTTCAGTGGCAGTGGATCTGGTACACAGTATTCTCTCAAGATAAA CAGCCTGCAGTCTGAAGATGTCGCAAGTTATTTCTGTCAACAGTATTACAATTATCC GTACACGTTTGGAGCTGGGACCAAGCTGGAACTGAAA (SEQ ID NO: 19). In some embodiments, the foregoing nucleotide sequences may be comprised within an expression vector.

In another aspect, the present disclosure provides methods of expanding or activating immune cells that express an anti-CD123 chimeric antigen receptor (CD123-CAR) comprising contacting in vitro a population of immune cells that express a CD123-CAR with an anti-idiotype antibody or antigen-binding fragment that specifically binds to an idiotype of the CD123-CAR, wherein the CD123-CAR comprises a scFv that binds to CD123, a hinge domain, a transmembrane domain, a co-stimulatory domain, and T cell receptor zeta chain signaling domain.

In another aspect, the present disclosure provides methods of detecting the presence of a CD123-CAR in a sample comprising, contacting a sample comprising immune cells that are suspected of expressing a CD123-CAR with an anti-idiotype antibody or antigen-binding fragment that specifically binds to an idiotype of the CD123-CAR and quantifying the number of cells expressing the CD123-CAR, wherein the CD123-CAR comprises a scFv that binds to CD123, a hinge domain, a transmembrane domain, at least one co-stimulatory domain, and T cell receptor zeta chain signaling domain. In some embodiments, the sample is a cell culture medium. In some embodiments, the sample is a blood sample from a subject that has been treated with the immune cells expressing the CD123-CAR. In some embodiments, the present method of detecting the presence of a CD123-CAR may further comprise recommending administration of further immune cells expressing the CD123-CAR if it is determined that the quantity of immune cells expressing the CD123-CAR is below a preset threshold. In some embodiments, the present method of detecting the presence of a CD123-CAR may further comprise administering further immune cells expressing the CD123-CAR if it is determined that the quantity of immune cells expressing the CD123-CAR is below a preset threshold. In some embodiments, the present method of detecting the presence of a CD123-CAR may further comprise recommending abstaining from administering further immune cells expressing the CD123-CAR if it is determined that the quantity of immune cells expressing the CD123-CAR is above a preset threshold. In some embodiments, the present method of detecting the presence of a CD123-CAR may further comprise abstaining from administering further immune cells expressing the CD123-CAR if it is determined that the quantity of immune cells expressing the CD123-CAR is above a preset threshold. In some embodiments, the present method of detecting the presence of a CD123-CAR may further comprise removing immune cells from the blood of the subject by contacting the blood with a solid support comprising an anti-idiotype antibody or antigen-binding fragment comprising, for example, a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYSN (SEQ ID NO: 4), a CDRL2 comprising DAS (SEQ ID NO: 5), and a CDRL3 comprising QQHHDYPLT (SEQ ID NO: 6); or a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYNG (SEQ ID NO: 7), a CDRL2 comprising NAN (SEQ ID NO: 8), and a CDRL3 comprising QQYYNYPYT (SEQ ID NO: 9); thereby sequestering immune cells that express the CD123-CAR from the blood of the subject, and subsequently administering the blood from which the immune cells that express the CD123-CAR were removed back to the subject. In some embodiments, the subject has acute myeloid leukemia (AML) blastic plasmacytoid dendritic cell neoplasm (BPDCN), acute lymphoblastic leukemia (ALL), or hairy cell leukemia.

In another aspect, the present disclosure provides methods of isolating immune cells that express an CD123-CAR from a sample comprising, contacting a sample comprising immune cells that are suspected of expressing an CD123-CAR with a solid support comprising an anti-idiotype antibody or antigen-binding fragment, thereby isolating the immune cells that express the CD123-CAR from the sample, wherein the CD123-CAR comprises a scFv that binds to CD123, a hinge domain, a transmembrane domain, a co-stimulatory domain, and T cell receptor zeta chain signaling domain. In some embodiments, the sample is a cell culture medium. In some embodiments, the sample is a blood sample from a subject that has been treated with the immune cells expressing the CD123-CAR. In some embodiments, the solid support may comprise a column or beads to which the anti-idiotype antibody or antigen-binding fragment is linked.

In some embodiments of the foregoing methods, the anti-idiotype antibody or antigen-binding fragment comprises: a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYSN (SEQ ID NO: 4), a CDRL2 comprising DAS (SEQ ID NO: 5), and a CDRL3 comprising QQHHDYPLT (SEQ ID NO: 6); or a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYNG (SEQ ID NO: 7), a CDRL2 comprising NAN (SEQ ID NO: 8), and a CDRL3 comprising QQYYNYPYT (SEQ ID NO: 9).

In some embodiments of the foregoing methods, the anti-idiotype antibody or antigen-binding fragment comprises: a V_{H} region comprising EVQLQQSGAELVRPGASVRLSCTTSG FNIKDSFIHWVKQRTEQGLEWIGRIDPEDDETKYAPKFQGKATITADTSSNTAYLQLSSL TSEDTAVYYCASPIYGSREAWFAYWGQGTLVTVSA (SEQ ID NO: 13) and a V_{L} comprising DIQMTQSPASLSASLGETVTIECLASEDIYSNLAWYQQKPGKSPQLLIYDASSLQDGVPS RFSGSESGTQYSLEINSLQSEDAATYFCQQHHDYPLTFGSGTKLEIK (SEQ ID NO: 14); or a V_{H} region comprising EVQLQQSGAELVRPGASVRLSCTTSGFNIKDSFIHWVKQR TEQGLEWIGRIDPEDDETKYAPKFQGKATITADTSSNTAYLQLSSLTSEDTAVYYCASPI YGSREAWFAYWGQGTLVTVSA (SEQ ID NO: 13) and a V_{L} comprising DIQMTQSPASLSASLGETVTIECRASEDIYNGLAWYQQKPGKSPQLLIYNANSLHTGVPS RFSGSGSGTQYSLKINSLQSEDVASYFCQQYYNYPYTFGAGTKLELK (SEQ ID NO: 15).

In some embodiments of the foregoing methods, the hinge of the CD123-CAR is an IgG hinge or a modified IgG hinge. In some embodiments of the foregoing methods, the co-stimulatory signaling domain of the CD123-CAR is selected from the group consisting of: a CD27 co-stimulatory signaling domain, a CD28 co-stimulatory signaling domain, a 4-1BB co-stimulatory signaling domain, and an OX40 co-stimulatory signaling domain. In some embodiments of the foregoing methods, the transmembrane domain comprises a transmembrane portion of CD28, CD4, CD8, 4-1BB, CD27, ICOS, OX40, HVEM, or CD30. In some embodiments of the foregoing methods, the scFv of the CD123-CAR comprises SEQ ID NOs: 20 and 21 or SEQ ID NOs: 22 and 23. In some embodiments of the foregoing methods, the CD123-CAR comprises SEQ ID NO: 49 or SEQ ID NO: 50. In some embodiments of the foregoing methods, the immune cell in which the CD123-CAR is express may be a T cell or a natural killer (NK) cell.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 shows the gating strategy for system suitability testing using flow cytometry.

FIG. 2 shows the gating strategy to define CAR+ cells using anti-idiotype staining.

FIG. 3 shows the disclosed anti-idiotype antibody specifically detects CD123-CAR T cells but not CS1-CAR T cells.

FIG. 4 shows the anti-idiotype antibody can be used to sensitively assess the presence of CD123-CARs or other anti-CD123 binding proteins comprising the same variable sequence. The sensitivity of detection was 1% for CAR+ cells.

FIG. 5 shows the sensitivity of the anti-idiotype antibody is maintained in blood samples.

### DETAILED DESCRIPTION

The present disclosure provides anti-idiotype antibodies that bind to the variable domain of anti-CD123 antibodies or fragments thereof or CD123-CARs. Accordingly, the presently disclosed anti-idiotype antibodies may be used to detect anti-CD123 antibodies or fragments and CD123-CAR T cells, distinguish T cells that express different CARs, and expand and/or activate CD123-CAR T cells. The detection and analytical capacity of the disclosed anti-idiotype antibodies will provide benefit to both the CAR T cell manufacturing process as well as the clinical application of CD123-CAR T cells.

### I. Definitions

It is to be understood that methods are not limited to the particular embodiments described, and as such may vary. It is also to be understood that the terminology used herein is for the purpose of describing particular embodiments only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means plus or minus 10% as well as the specified number. For example, “about 10” should be understood as both “10” and “9-11.”

As used herein, “optional” or “optionally” means that the subsequently described event or circumstance may or may not occur, and that the description includes instances where said event or circumstance occurs and instances where it does not.

As used herein, the terms “individual”, “patient”, or “subject” can be an individual organism, a vertebrate, a mammal (e.g., a bovine, a canine, a feline, or an equine), or a human. In a preferred embodiment, the individual, patient, or subject is a human.

As used herein, the term an “isolated antibody” is intended to refer to an antibody which is substantially free of other antibodies having different antigenic specificities (e.g., an isolated anti-idiotype antibody is substantially free of antibodies that do not bind to the idiotype on an anti-CD123 antibody or CD123-CAR). An isolated antibody that specifically binds to an idiotype of an anti-CD123 antibody or CD123-CAR may, however, have cross-reactivity to other proteins. However, the antibody preferably always binds to an idiotype of an anti-CD123 antibody or CD123-CAR. In addition, an isolated antibody is typically substantially free of other cellular material and/or chemicals.

As used herein, the term “humanized antibody” refers to an antibody that comprises the CDRs of antibodies derived from mammals other than human, and the framework (FR) region and the constant region of a human antibody. A humanized antibody is useful as an effective component in a therapeutic agent according to the present disclosure since antigenicity of the humanized antibody in human body is lowered.

As used herein, the term “recombinant human antibody” includes all human antibodies that are prepared, expressed, created or isolated by recombinant means, including but not limited to (a) antibodies isolated from an animal (e.g., a mouse) that is transgenic or transchromosomal for human immunoglobulin genes or a hybridoma prepared therefrom, (b) antibodies isolated from a host cell transformed to express the antibody (e.g., from a transfectoma), (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline and/or non-germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, the term “glycosylation pattern” is defined as the pattern of carbohydrate units that are covalently attached to a protein, more specifically to an immunoglobulin protein.

As used herein, the phrases “therapeutically effective amount” and “therapeutic level” mean that drug dosage or plasma concentration in a subject, respectively, that provides the specific pharmacological effect for which the drug is administered in a subject in need of such treatment, i.e. to reduce, ameliorate, or eliminate the symptoms or effects of cancer, malignant disease, or cancer cell proliferation. It is emphasized that a therapeutically effective amount or therapeutic level of a drug will not always be effective in treating the conditions/diseases described herein, even though such dosage is deemed to be a therapeutically effective amount by those of skill in the art. The therapeutically effective amount may vary based on the route of administration and dosage form, the age and weight of the subject, and/or the subject's condition, including the type and stage of the cancer, malignant disease, or cancer cell proliferation, among other factors.

The terms “treatment” or “treating” as used herein with reference to cancer, malignant disease, or cancer cell proliferation refer to reducing, ameliorating or eliminating one or more symptoms or effects of cancer, malignant disease, or cancer cell proliferation.

### II. Anti-Idiotype Antibodies

Provided herein are anti-idiotype antibodies that bind to the variable domain of a CD123-binding protein or peptide. The disclosed anti-idiotype antibodies and functional fragments thereof will have a variety of functional properties for detecting and assessing CD123-binding proteins or peptide, such as an anti-CD123 antibody or CD123-CAR.

The disclosed anti-idiotype antibodies can be polyclonal, monoclonal, chimeric, human, partially or fully humanized, and/or recombinant.

Polyclonal antibodies may be obtained by methods known in the art, such as by immunizing a selected animal with an antigen comprising all of part of the variable domain of an anti-CD123 antibody or CD123-CAR, collecting serum from the animal, and isolating and/or purifying antibodies from the serum. Monoclonal antibodies (mAbs) may be obtained by methods known in the art, for example, by fusing antibody-producing cells with immortalized cells to obtain a hybridoma, and/or by generating mAbs from mRNA extracted from bone marrow and spleen cells of immunized animals using combinatorial antibody library technology. Recombinant antibodies may be obtained by methods known in the art, for example, using phage or yeast display technologies and/or expressing or co-expressing antibody polypeptides. Other techniques for making antibodies are known in the art, and can be used to obtain antibodies used in the methods described herein. The disclosed antibodies may be derived from a suitable animal, including but not limited to a rat, mouse, pig, goat, bovine, horse, or human.

Typically, an antibody consists of four polypeptides: two identical copies of a heavy (H) chain polypeptide and two copies of a light (L) chain polypeptide. Typically, each heavy chain contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody.

The terms “binding fragment” or “functional fragment,” as used herein, refer to one or more fragments of an anti-idiotype antibody that retains the ability to bind the variable domain of an anti-CD123 antibody or CD123-CAR. Examples of binding fragments include (i) Fab fragments (monovalent fragments consisting of the VL, VH, CL and CH1 domains); (ii) F(ab′)2 fragments (bivalent fragment comprising two Fab fragments linked by a disulfide bridge at the hinge region); (iii) Fd fragments (comprising the VH and CH1 domains); (iv) Fv fragments (comprising the VL and VH domains of a single arm of an antibody), (v) dAb fragments (comprising a VH domain); and (vi) isolated complementarity determining regions (CDR), e.g., VH CDR3. Other examples include single chain Fv (scFv) constructs. See e.g., Bird et al., Science, 242:423-26 (1988); Huston et al., Proc. Natl. Acad. Sci. USA, 85:5879-83 (1988).

In some embodiments, the anti-idiotype antibody or a fragment thereof may comprise the exemplary CDR sequences disclosed in Tables 1 and 2 below.

TABLE 1

| Anti- | | | |
|---|---|---|---|
| body | HCDR1 | HCDR2 | HCDR3 |
| 1 | GFNIKDSF | IDPEDDET | ASPIYGSREAWFAY |
| | (SEQ ID | (SEQ ID | (SEQ ID NO: 3) |
| | NO: 1) | NO: 2) | |
| 2 | GFNIKDSF | IDPEDDET | ASPIYGSREAWFAY |
| | (SEQ ID | (SEQ ID | (SEQ ID NO: 3) |
| | NO: 1) | NO: 2) | |

TABLE 2

| Anti- | | | |
|---|---|---|---|
| body | LCDR1 | LCDR2 | LCDR3 |
| 1 | EDIYSN | DAS | QQHHDYPLT |
| | (SEQ ID | (SEQ ID | (SEQ ID |
| | NO: 4) | NO: 5) | NO: 6) |
| 2 | EDIYNG | NAN | QQYYNYPYT |
| | (SEQ ID | (SEQ ID | (SEQ ID |
| | NO: 7) | NO: 8) | NO: 9) |

It should be understood that substitutions or other alterations to the CDR sequences disclosed in Tables 1 and 2 may permitted while still allowing the anti-idiotype antibody to bind the variable domain of, for example, an anti-CD123 antibody or fragment thereof. Accordingly, some of the amino acids in the CDR sequences are variable and may be changed.

For example, in some embodiments, the CDRL1 of the disclosed anti-idiotype antibodies may comprise EDIYX₁X₂ (SEQ ID NO: 10), wherein X₁ is a polar amino acid; and wherein X₂ is selected from the group consisting of serine (S), threonine (T), asparagine (N), glutamine (Q), cysteine (C), glycine (G), and proline (P). In some embodiments, X₁ is serine (S) or asparagine (N). In some embodiments, X₂ is asparagine (N) or glycine (G).

In some embodiments, the CDRL2 of the disclosed anti-idiotype antibodies may comprise X₃AX₄ (SEQ ID NO: 11), wherein X₃ is selected from the group consisting of aspartic acid (D), glutamic acid (E), serine (S), threonine (T), asparagine (N), and glutamine (Q); and wherein X₄ is a polar amino acid. In some embodiments, X₃ is aspartic acid (D) or asparagine (N). In some embodiments, X₄ is serine (S) or asparagine (N).

In some embodiments, the CDRL3 of the disclosed anti-idiotype antibodies may comprise QQX₅X₆X₇YPX₈T (SEQ ID NO: 12), wherein X₅ and X₆ are independently selected from the group consisting of arginine (R), histidine (H), lysine (K), alanine (A), valine (V), isoleucine (I), leucine (L), methionine (M), phenylalanine (F), tyrosine (Y), and tryptophan (W); X₇ is selected from the group consisting of aspartic acid (D), glutamic acid (E), serine (S), threonine (T), asparagine (N), and glutamine (Q); and X₈ is selected from the group consisting of alanine (A), valine (V), isoleucine (I), leucine (L), methionine (M), phenylalanine (F), tyrosine (Y), and tryptophan (W). In some embodiments, X₅ is histidine (H) or tyrosine (Y). In some embodiments, X₆ is histidine (H) or tyrosine (Y). In some embodiments, X₇ is aspartic acid (D) or asparagine (N). In some embodiments, X₈ is leucine (L) or tyrosine (Y).

In some embodiments, of the disclosed anti-idiotype antibodies X₁ is serine (S) or asparagine (N); X₂ is asparagine (N) or glycine (G); X₃ is aspartic acid (D) or asparagine (N); wherein X₄ is serine (S) or asparagine (N); X₅ and X₆ are histidine (H) or tyrosine (Y); X₇ is aspartic acid (D) or asparagine (N); and X₈ is leucine (L) or tyrosine (Y).

Some embodiments of the disclosed anti-idiotype antibodies may comprise a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYSN (SEQ ID NO: 4), a CDRL2 comprising DAS (SEQ ID NO: 5), and a CDRL3 comprising QQHHDYPLT (SEQ ID NO: 6); or a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYNG (SEQ ID NO: 7), a CDRL2 comprising NAN (SEQ ID NO: 8), and a CDRL3 comprising QQYYNYPYT (SEQ ID NO: 9).

The disclosed anti-idiotype antibodies or binding fragments may also comprise a variable heavy chain domain (V_{H}) and a variable light chain domain (V_{L}). For example, the V_{H} region of the anti-idiotype antibody or fragment thereof may comprise:

EVQLQQSGAELVRPGASVRLSCTTSGFNIKDSFIHWVKQRTEQGLEWIGRIDPEDDETKY APKFQGKATITADTSSNTAYLQLSSLTSEDTAVYYCASPIYGSREAWFAYWGQGTLVTV SA (SEQ ID NO: 13); and the V_{L} region of the anti-idiotype antibody or fragment thereof may comprise:

(SEQ ID NO: 14)
DIQMTQSPASLSASLGETVTIECLASEDIYSNLAWYQQKPGKSPQLL
IYDASSLQDGVPSRFSGSESGTQYSLEINSLQSEDAATYFCQQHHDY
PLTFGSGTKLEIK;
or
(SEQ ID NO: 15)
DIQMTQSPASLSASLGETVTIECRASEDIYNGLAWYQQKPGKSPQLL
IYNANSLHTGVPSRFSGSGSGTQYSLKINSLQSEDVASYFCQQYYNY
PYTFGAGTKLELK.

Additionally, in some embodiments, the disclosed antibodies and fragments may comprise various modifications (i.e., substitutions, additions, or deletions) to their framework regions. Indeed, the disclosed CDRs and variable regions can be readily adapted to various Fc formats, including, for example, a human, mouse, or rat IgG such as IgG2a. In some embodiments, the anti-idiotype antibody may be biotinylated or non-biotinylated.

The disclosed antibodies or antigen-binding fragments may be encoded by one or more of the nucleic acid sequences shown in Table 3 below. The nucleic acid sequences encoding the disclosed antibodies and fragments may be incorporated into, e.g., an expression vector to allow for recombinant expression of the disclosed antibodies or fragments.

TABLE 3

| SEQ | | |
|---|---|---|
| ID | | |
| NO | Description | Sequence |
| 16 | V_{H} encoding | GAGGTTCAGCTGCAGCAGTCTGGGGCAGAGCT |
| | sequence 1 | TGTGAGGCCAGGGGCCTCAGTCAGGTTGTCCT |
| | | GCACAACTTCTGGCTTCAACATTAAAGACTCC |
| | | TTTATTCACTGGGTGAAGCAGAGGACTGAACA |
| | | GGGCCTGGAGTGGATTGGAAGGATTGATCCTG |
| | | AGGATGATGAAACTAAATATGCCCCGAAATTC |
| | | CAGGGCAAGGCCACTATAACAGCAGACACATC |
| | | CTCCAACACAGCCTACCTGCAGCTCAGCAGCC |
| | | TGACATCTGAGGACACTGCCGTCTATTACTGT |
| | | GCTAGCCCCATCTACGGTAGTAGAGAGGCCTG |
| | | GTTTGCTTACTGGGGCCAAGGGACTCTGGTCA |
| | | CTGTCTCTGCA |
| 17 | V_{L} encoding | GACATCCAGATGACACAGTCTCCAGCTTCCCT |
| | sequence 1 | GTCTGCATCTCTGGGAGAAACTGTCACCATCG |
| | | AATGTCTAGCAAGTGAAGACATTTACAGTAAT |
| | | TTAGCGTGGTATCAGCAGAAGCCAGGGAAATC |
| | | TCCTCAGCTCCTGATCTATGATGCAAGTAGCT |
| | | TGCAAGATGGGGTCCCATCACGGTTCAGTGGC |
| | | AGTGAATCTGGCACACAGTATTCTCTCGAGAT |
| | | CAACAGCCTGCAATCTGAAGATGCCGCGACTT |
| | | ATTTCTGTCAACAGCATCATGATTATCCTCTC |
| | | ACGTTCGGTTCTGGGACCAAGCTGGAGATCAA |
| | | A |
| 18 | V_{H} encoding | GAGGTTCAGCTGCAGCAGTCTGGGGCAGAGCT |
| | sequence 2 | TGTGAGGCCAGGGGCCTCAGTCAGGTTGTCCT |
| | | GCACAACTTCTGGCTTCAACATTAAAGACTCC |
| | | TTTATTCACTGGGTGAAGCAGAGGACTGAACA |
| | | GGGCCTGGAGTGGATTGGAAGGATTGATCCTG |
| | | AGGATGATGAAACTAAATATGCCCCGAAATTC |
| | | CAGGGCAAGGCCACTATAACAGCAGACACATC |
| | | CTCCAACACAGCCTACCTGCAGCTCAGCAGCC |
| | | TGACATCTGAGGACACTGCCGTCTATTACTGT |
| | | GCTAGCCCCATCTACGGTAGTAGAGAGGCCTG |
| | | GTTTGCTTACTGGGGCCAAGGGACTCTGGTCA |
| | | CTGTCTCTGCA |
| 19 | V_{L} encoding | GACATCCAGATGACACAGTCTCCAGCTTCCCT |
| | sequence 2 | GTCTGCATCTCTGGGAGAAACTGTCACCATCG |
| | | AATGTCGAGCAAGTGAGGACATTTACAATGGT |
| | | TTAGCATGGTATCAGCAGAAGCCAGGGAAATC |
| | | TCCTCAGCTCCTGATCTATAATGCAAATAGCT |
| | | TGCATACTGGGGTCCCATCACGGTTCAGTGGC |
| | | AGTGGATCTGGTACACAGTATTCTCTCAAGAT |
| | | AAACAGCCTGCAGTCTGAAGATGTCGCAAGTT |
| | | ATTTCTGTCAACAGTATTACAATTATCCGTAC |
| | | ACGTTTGGAGCTGGGACCAAGCTGGAACTGAA |
| | | A |

The disclosed antibodies or antigen-binding fragments specifically bind to an idiotype on an anti-CD123 antibody or an anti-CD123 antigen-binding fragment. For example, the disclosed antibodies or antigen-binding fragments may specifically bind to an anti-CD123 antibody or anti-CD123 antigen-binding fragment (e.g., a scFv) that comprises the V_{L} domain of DVQITQSPSYLAASPGETITINCRASKSISKDLAWYQEKPGKTNKLLIYSGSTLQSGIPSRF SGSGSGTDFTLTISSLEPEDFAMYYCQQHNKYPYTFGGGTKLEIK (SEQ ID NO: 20) and/or the V_{H} domain of

QVQLQQPGAELVRPGASVKLSCKASGYTFTSYWMNWVKQRPDQGLEWIGRIDPYDSET HYNQKFKDKAILTVDKSSSTAYMQLSSLTSEDSAVYYCARGNWDDYWGQGTTLTVSS (SEQ ID NO: 21). Alternatively, the disclosed antibodies or antigen-binding fragments may specifically bind to an anti-CD123 antibody or anti-CD123 antigen-binding fragment (e.g., a scFv) that comprises the V_{L} domain of
DIVLTQSPASLAVSLGQRATISCRASESVDNYGNTFMHWYQQKPGQPPKLLIYRASNLES GIPARFSGSGSRTDFTLTINPVEADDVATYYCQQSNEDPPTFGAGTKLELK (SEQ ID NO: 22) and/or the V_{H} domain of
QIQLVQSGPELKKPGETVKISCKASGYIFTNYGMNWVKQAPGKSFKWMGWINTYTGES TYSADFKGRFAFSLETSASTAYLHINDLKNEDTATYFCARSGGYDPMDYWGQGTSVTV SS (SEQ ID NO: 23). The anti-CD123 antigen-binding fragment may be an isolated fragment or it may be incorporated into a larger construct, such as a chimeric antigen receptor (CAR).

In general, a CD123-CAR that can be bound by the disclosed anti-idiotype antibodies and fragments may comprise (a) a hinge and/or linker, such as an IgG hinge or a modified IgG hinge, (b) a transmembrane domain, (c) one or more co-stimulatory signaling domain(s), and (d) a T cell receptor zeta chain signaling domain (e.g., a CD3ζ domain).

Those of skill in the art will understand that the co-stimulatory signaling domain(s) may be selected from, for example, the group consisting of: a CD27 co-stimulatory signaling domain, a CD28 co-stimulatory signaling domain (or a modified CD28 domain), a 4-1BB co-stimulatory signaling domain, and an OX40 co-stimulatory signaling domain, or a combination thereof. Some CARs may comprise one co-stimulatory domain, while others may contain two or three. Exemplary costimulatory domains are provided in the following table.

TABLE 4
Exemplary Costimulatory Domains

| SEQ ID | Descrip- | |
|---|---|---|
| NO: | tion | Sequence |
| 24 | CD28 | RSKRSRLLHSDYMNMTPRRPGPTR |
| | | KHQYPYAPPRDFAAYRS |
| 25 | CD28gg | RSKRSRGGHSDYMNMTPRRPGPTR |
| | | KHYQPYAPPRDFAAYRS |
| 26 | 4-1BB | KRGRKKLLYIFKQPFMRPVQTTQE |
| | | EDGCSCRFPEEEEGGCEL |
| 27 | OX40 | ALYLLRRDQRLPPDAHKPPGGGSF |
| | | RTPIQEEQADAHSTLAKI |

Those of skill in the art will understand that the transmembrane domain may be selected from, for example, a transmembrane portion of CD28, CD4, CD8, 4-1BB, CD27, ICOS, OX40, HVEM, or CD30. Exemplary transmembrane domains are provided in the following table.

TABLE 5
Exemplary Transmembrane Domains

| SEQ ID | Descrip- | |
|---|---|---|
| NO: | tion | Sequence |
| 28 | CD3z | LCYLLDGILFIYGVILTALFL |
| 29 | CD28 | FWVLVVVGGVLACYSLLVTVAFIIFWV |
| 30 | CD28(M) | MFWVLVVVGGVLACYSLLVTVAFIIFWV |
| 31 | CD4 | MALIVLGGVAGLLLFIGLGIFF |
| 32 | CD8(i) | IYIWAPLAGTCGVLLLSLVIT |
| 33 | CD8(ii) | IYIWAPLAGTCGVLLLSLVITLY |
| 34 | CD8(iii) | IYIWAPLAGTCGVLLLSLVITLYC |
| 35 | 4-1BB | IISFFLALTSTALLFLLFFLTLRF |

Those of skill in the art will understand that the hinge or linker may be selected from, for example, an IgG4 hinge or derivative thereof, an IgG2 hinge or derivative thereof, a CD28 hinge, or a CD8 hinge, or another suitable peptide linker, such as a G or S repeat. Exemplary hinges/linkers domains are provided in the following table.

TABLE 6
Exemplary Linkers and Hinges

| SEQ ID | | |
|---|---|---|
| NO: | Description | Sequence |
| 36 | A3 | AAA |
| 37 | Linker | GGGSSGGGSG |
| 38 | IgG4 hinge | ESKYGPPCPPCP |
| | (S228P) | |
| 39 | IgG4 hinge | ESKYGPPCPSCP |
| 40 | IgG4 hinge + | ESKYGPPCPPCPGGGSSGGGSG |
| | linker | |
| 41 | CD28 hinge | IEVMYPPPYLDNEKSNGTIIHVK |
| | | GKHLCPSPLFPGPSKP |
| 42 | CD8 hinge | AKPTTTPAPRPPTPAPTIASQPL |
| | (48 AA) | SLRPEACRPAAGGAVHTRGLDF |
| | | ACD |
| 43 | CD8 hinge | TTTPAPRPPTPAPTIASQPLSLR |
| | (45 AA) | PEACRPAAGGAVHTRGLDFACD |
| 44 | IgG4 | ESKYGPPCPPCPGGGSSGGGSGG |
| | (HL-CH3) | QPREPQVYTLPPSQEEMTKNQVS |
| | | LTCLVKGFYPSDIAVEWESNGQP |
| | | ENNYKTTPPVLDSDGSFFLYSRL |
| | | TVDKSRWQEGNVFSCSVMHEALH |
| | | NHYTQKSLSLSLGK |
| 45 | IgG4 (L235E, | ESKYGPPCPSCPAPEFEGGPSVF |
| | N297Q) | LFPPKPKDTLMISRTPEVTCVVV |
| | | DVSQEDPEVQFNWYVDGVEVHNA |
| | | KTKPREEQFQSTYRVVSVLTVLH |
| | | QDWLNGKEYKCKVSNKGLPSSIE |
| | | KTISKAKGQPREPQVYTLPPSQE |
| | | EMTKNQVSLTCLVKGFYPSDIAV |
| | | EWESNGQPENNYKTTPPVLDSDG |
| | | SFFLYSRLTVDKSRWQEGNVFSC |
| | | SVMHEALHNHYTQKSLSLSLGK |
| 46 | IgG4 (S228P, | ESKYGPPCPPCPAPEFEGGPSVF |
| | L235E, N297Q) | LFPPKPKDTLMISRTPEVTCVVV |
| | | DVSQEDPEVQFNWYVDGVEVHNA |
| | | KTKPREEQFQSTYRVVSVLTVLH |
| | | QDWLNGKEYKCKVSNKGLPSSIE |
| | | KTISKAKGQPREPQVYTLPPSQE |
| | | EMTKNQVSLTCLVKGFYPSDIAV |
| | | EWESNGQPENNYKTTPPVLDSDG |
| | | SFFLYSRLTVDKSRWQEGNVFSC |
| | | SVMHEALHNHYTQKSLSLSLGK |
| 47 | IgG4 (CH3) | GQPREPQVYTLPPSQEEMTKNQV |
| | | SLTCLVKGFYPSDIAVEWESNGQ |
| | | PENNYKTTPPVLDSDGSFFLYSR |
| | | LTVDKSRWQEGNVFSCSVMHEAL |
| | | HNHYTQKSLSLSLGK |

In some embodiments, the CD123-CAR may comprise a CD3ζ signaling domain

(RVKFSRSADAPAYQQGQNQLYNELNLGRREEYDVLDKRRGRDPEMG
GKPRRKNPQEGLYNELQKDKMAEAYSEIGMKGERRRGKGHDGLYQGL
STATKDTYDALHMQALPPR; SEQ ID NO: 48).

The antibody or antigen-binding fragment of claim 21, wherein the CD123 CAR comprises an amino acid sequence:

(SEQ ID NO: 49)
QVQLQQPGAELVRPGASVKLSCKASGYTFTSYWMNWVKQRPDQGLEW
IGRIDPYDSETHYNQKFKDKAILTVDKSSSTAYMQLSSLTSEDSAVY
YCARGNWDDYWGQGTTLTVSSGGGGSGGGGSGGGGSDVQITQSPSYL
AASPGETITINCRASKSISKDLAWYQEKPGKTNKLLIYSGSTLQSGI
PSRFSGSGSGTDFTLTISSLEPEDFAMYYCQQHNKYPYTFGGGTKLE
IKESKYGPPCPPCPAPEFEGGPSVFLFPPKPKDTLMISRTPEVTCVV
VDVSQEDPEVQFNWYVDGVEVHNAKTKPREEQFQSTYRVVSVLTVLH
QDWLNGKEYKCKVSNKGLPSSIEKTISKAKGQPREPQVYTLPPSQEE
MTKNQVSLTCLVKGFYPSDIAVEWESNGQPENNYKTTPPVLDSDGSF
FLYSRLTVDKSRWQEGNVFSCSVMHEALHNHYTQKSLSLSLGKMFWV
LVVVGGVLACYSLLVTVAFIIFWVRSKRSRGGHSDYMNMTPRRPGPT
RKHYQPYAPPRDFAAYRSGGGRVKFSRSADAPAYQQGQNQLYNELNL
GRREEYDVLDKRRGRDPEMGGKPRRKNPQEGLYNELQKDKMAEAYSE
IGMKGERRRGKGHDGLYQGLSTATKDTYDALHMQALPPR;
or
(SEQ ID NO: 50)
QIQLVQSGPELKKPGETVKISCKASGYIFTNYGMNWVKQAPGKSFKW
MGWINTYTGESTYSADFKGRFAFSLETSASTAYLHINDLKNEDTATY
FCARSGGYDPMDYWGQGTSVTVSSGGGGSGGGGSGGGGSDIVLTQSP
ASLAVSLGQRATISCRASESVDNYGNTFMHWYQQKPGQPPKLLIYRA
SNLESGIPARFSGSGSRTDFTLTINPVEADDVATYYCQQSNEDPPTF
GAGTKLELKESKYGPPCPPCPAPEFEGGPSVFLFPPKPKPKDTLMIS
RTPEVTCVVVDVSQEDPEVQFNWYVDGVEVHNAKTKPREEQFQSTYR
VVSVLTVLHQDWLNGKEYKCKVSNKGLPSSIEKTISKAKGQPREPQV
YTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLS
LSLGKMFWVLVVVGGVLACYSLLVTVAFIIFWVRSKRSRGGHSDYMN
MTPRRPGPTRKHYQPYAPPRDFAAYRSGGGRVKFSRSADAPAYQQGQ
NQLYNELNLGRREEYDVLDKRRGRDPEMGGKPRRKNPQEGLYNELQK
DKMAEAYSEIGMKGERRRGKGHDGLYQGLSTATKDTYDALHMQALPP
R.

A CD123-CAR may optionally comprise additional components, such as a leader sequence or a surrogate tag. An exemplary leader sequence is MLLLVTSLLLCELPHPAFLLIP (SEQ ID NO: 51). Surrogate tags include, but are not limited to, truncated proteins such as CD19, epidermal growth factor receptor (EGFR), CD3.4, and NGFR, which can be used to identify cells that have been transformed with a nucleic acid for expressing a CAR (e.g., a CD123-CAR). Exemplary surrogate tag sequences include truncated EGFR (“EGFRt”):

MLLLVTSLLLCELPHPAFLLIPRKVCNGIGIGEFKDSLSINATNIKHFKNCTSISGDLHILPV AFRGDSFTHTPPLDPQELDILKTVKEITGFLLIQAWPENRTDLHAFENLEIIRGRTKQHGQ FSLAVVSLNITSLGLRSLKEISDGDVIISGNKNLCYANTINWKKLFGTSGQKTKIISNRGEN SCKATGQVCHALCSPEGCWGPEPRDCVSCRNVSRGRECVDKCNLLEGEPREFVENSECI QCHPECLPQAMNITCTGRGPDNCIQCAHYIDGPHCVKTCPAGVMGENNTLVWKYADA GHVCHLCHPNCTYGCTGPGLEGCPTNGPKIPSIATGMVGALLLLLVVALGIGLFMGMCS FRA SIGNAL PEPTIDE (SEQ ID NO: 52), and truncated CD19 (“CD19t”):
MPPPRLLFFLLFLTPMEVRPEEPLVVKVEEGDNAVLQCLKGTSDGPTQQLTWSRESPLKP FLKLSLGLPGLGIHMRPLAIWLFIFNVSQQMGGFYLCQPGPPSEKAWQPGWTVNVEGSG ELFRWNVSDLGGLGCGLKNRSSEGPSSPSGKLMSPKLYVWAKDRPEIWEGEPPCVPPRD SLNQSLSQDLTMAPGSTLWLSCGVPPDSVSRGPLSWTHVHPKGPKSLLSLELKDDRPAR DMWVMETGLLLPRATAQDAGKYYCHRGNLTMSFHLEITARPVLWHWLLRTGGWKVS AVTLAYLIFCLCSLVGILHLQRALVLRRKR (SEQ ID NO: 53). Surrogate tags are commonly linked to CARs via a cleavage T2A linker: LEGGGEGRGSLLTCGDVEENPGPR (SEQ ID NO: 54).

Surrogate tags are commonly used for quality control purposes, but they present several drawbacks. For example, surrogate tags can identify cells that express a CAR and the surrogate tag, but they cannot differentiate between CARs. According, this may present problems in a facility where multiple types of CAR-expressing cells are produced and must be distinguished. The disclosed anti-idiotype antibodies and fragments are CAR-specific, rather than cell-specific, and allow an artisan to distinguish between multiple CARs even if those CARs are expressed in cells that carry the same surrogate tag.

One of ordinary skill in the art will understand that certain changes can be made to the disclosed sequences without compromising the binding affinity or function of the disclosed anti-idiotype antibodies and functional fragments. According, in some embodiments, the anti-idiotype antibodies or fragments will share about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the disclosed sequences (e.g., SEQ ID NOs: 1-9 and 13-15).

In some embodiments, the disclosure provides for isolated nucleic acid sequences encoding an anti-idiotype antibody or fragment thereof, for example, SEQ ID NOs: 1-9 and 13-15.

The disclosed anti-idiotype antibodies and fragments thereof can be formulated in a pharmaceutical composition suitable for administration to the target subject; immobilized on a solid support for various diagnostic, quality assurance, or clinical application; or formulated for in vitro use in detection of transduced cells or as an activator of transduced cells.

### III. Methods of Using the Disclose Anti-Idiotype Antibodies or Fragments

The disclosed anti-idiotype antibodies and fragments are useful for a variety of manufacturing, quality control, diagnostic, and clinical applications.

In one aspect, the disclosed anti-idiotype antibodies and fragments may be used to detect the presence of a CD123-CAR or anti-CD123 antibody in a sample by contacting the sample with an anti-idiotype antibody or antigen-binding fragment that specifically binds to an idiotype of the CD123-CAR or anti-CD123 antibody and quantifying amount of bound anti-idiotype antibody. Such a methods allows for quantification of the number of cells expressing a CD123-CAR or an anti-CD123 antibody. For the purposes of the detection and quantification methods, the disclosed anti-idiotype antibodies may be used in an ELISA format or in a flow cytometry assay to detect and/or quantify a CD123-CAR or anti-CD123 antibody. In some embodiments of the disclosed detection and quantification methods, the disclosed anti-idiotype antibodies may be used for immunohistochemistry.

The sample may be a cell culture medium (e.g., in the case of quantification during manufacturing or expansion of CD123-CAR expressing cells), or the sample may a blood sample from a subject that has been treated with the immune cells expressing the CD123-CAR (e.g., to determine the persistence of the cells in the patent's circulation or to determine whether the patient has received a sufficient dose).

For the purposes of clinical applications in which the disclosed anti-idiotype antibodies or fragments are used to determine the persistence or relative dose of CD123-CAR cells in a blood sample from a patient, the methods may further comprise recommending administration of further immune cells expressing the CD123-CAR if it is determined that the quantity of immune cells expressing the CD123-CAR is below a preset threshold. In contrast, such a method may further comprise recommending abstaining from administering further immune cells expressing the CD123-CAR if it is determined that the quantity of immune cells expressing the CD123-CAR is above a preset threshold. Alternatively, the method may further comprise removing immune cells that express the CD123-CAR from the blood of the subject by contacting the blood with a solid support comprising an anti-idiotype antibody or fragment thereof comprising, for example, a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYSN (SEQ ID NO: 4), a CDRL2 comprising DAS (SEQ ID NO: 5), and a CDRL3 comprising QQHHDYPLT (SEQ ID NO: 6); or a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYNG (SEQ ID NO: 7), a CDRL2 comprising NAN (SEQ ID NO: 8), and a CDRL3 comprising QQYYNYPYT (SEQ ID NO: 9); thereby sequestering immune cells that express the CD123-CAR from the blood of the subject, and subsequently administering the blood from which the immune cells that express the CD123-CAR were removed back to the subject. CD123 is a target that is being utilized in the treatment of acute myeloid leukemia (AML), and therefore, in some embodiments of such clinical applications, the patient may have or be suspected of having AML. CD123, however, may also be a useful target in other hematological cancers or conditions such as blastic plasmacytoid dendritic cell neoplasm (BPDCN), acute lymphoblastic leukemia (ALL), or hairy cell leukemia.

Additionally, the disclosed anti-idiotype antibodies and fragments may be used for isolating immune cells that express a CD123-CAR from a sample by contact a sample comprising immune cells that are suspected of expressing an CD123-CAR with a solid support comprising an anti-idiotype antibody or antigen-binding fragment, thereby isolating the immune cells that express the CD123-CAR from the sample. In some embodiments, the sample may be a cell culture medium, while in some embodiments, the sample may be a blood sample from a subject that has been treated with the immune cells expressing the CD123-CAR. In some embodiments, the solid support may comprise a column or beads to which the anti-idiotype antibody or fragment is linked.

During the manufacture of CAR-expressing cells, such as T cell or natural killer (NK) cells, one of the production steps is to activate the CARs that are expressed by the cells in order to expand the transduced cell population. The process of activation and expansion may comprise contacting the CAR-expressing cells with a ligand for the CAR. The disclosed anti-idiotype antibodies or fragments bind to the variable domain of a CD123-CAR, and therefore may agonize the receptor, thus activating the T cell and expanding the CD123-CAR expressing population. According, the present disclosure provides methods of activating and/or expanding a population of CD123-CAR expressing cells (e.g., T cells or NK cells) comprising contacting a population of CD123-CAR expressing cells in vitro with the disclosed antibodies or fragments thereof.

In some embodiments of the foregoing methods, the anti-idiotype antibody or antigen-binding fragment comprises: a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYSN (SEQ ID NO: 4), a CDRL2 comprising DAS (SEQ ID NO: 5), and a CDRL3 comprising QQHHDYPLT (SEQ ID NO: 6); or a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYNG (SEQ ID NO: 7), a CDRL2 comprising NAN (SEQ ID NO: 8), and a CDRL3 comprising QQYYNYPYT (SEQ ID NO: 9).

In some embodiments of the foregoing methods, the anti-idiotype antibody or antigen-binding fragment comprises: a V_{H} region comprising EVQLQQSGAELVRPGASVRLSCTTSG FNIKDSFIHWVKQRTEQGLEWIGRIDPEDDETKYAPKFQGKATITADTSSNTAYLQLSSL TSEDTAVYYCASPIYGSREAWFAYWGQGTLVTVSA (SEQ ID NO: 13) and a V_{L} comprising DIQMTQSPASLSASLGETVTIECLASEDIYSNLAWYQQKPGKSPQLLIYDASSLQDGVPS RFSGSESGTQYSLEINSLQSEDAATYFCQQHHDYPLTFGSGTKLEIK (SEQ ID NO: 14); or a V_{H} region comprising EVQLQQSGAELVRPGASVRLSCTTSGFNIKDSFIHWVKQR TEQGLEWIGRIDPEDDETKYAPKFQGKATITADTSSNTAYLQLSSLTSEDTAVYYCASPI YGSREAWFAYWGQGTLVTVSA (SEQ ID NO: 13) and a V_{L} comprising DIQMTQSPASLSASLGETVTIECRASEDIYNGLAWYQQKPGKSPQLLIYNANSLHTGVPS RFSGSGSGTQYSLKINSLQSEDVASYFCQQYYNYPYTFGAGTKLELK (SEQ ID NO: 15).

In some embodiments of the foregoing methods, the hinge of the CD123-CAR is an IgG hinge or a modified IgG hinge. In some embodiments of the foregoing methods, the co-stimulatory signaling domain of the CD123-CAR is selected from the group consisting of: a CD27 co-stimulatory signaling domain, a CD28 co-stimulatory signaling domain, a 4-1BB co-stimulatory signaling domain, and an OX40 co-stimulatory signaling domain. In some embodiments of the foregoing methods, the transmembrane domain comprises a transmembrane portion of CD28, CD4, CD8, 4-1BB, CD27, ICOS, OX40, HVEM, or CD30. In some embodiments of the foregoing methods, the scFv of the CD123-CAR comprises SEQ ID NOs: 20 and 21 or SEQ ID NOs: 22 and 23. In some embodiments of the foregoing methods, the CD123-CAR comprises SEQ ID NO: 49 or SEQ ID NO: 50. In some embodiments of the foregoing methods, the immune cell in which the CD123-CAR is expressed may be a T cell or a natural killer (NK) cell.

The following examples are given to illustrate the present invention. It should be understood, however, that the invention is not to be limited to the specific conditions or details described in these examples.

### EXAMPLES

### Example 1—Methods for Differentiating CD123-CAR T Cells In Vitro

Introduction

This protocol was intended to qualify the identity method for release of a CD123-CAR expressing cell product using an anti-idiotype antibody and flow cytometry. This assay used an anti-idiotype antibody, which is directed specifically against the idiotype on the CD123 CAR.

Objective

This qualification evaluated the flow cytometry-based identity assay for use with a CD123-CAR expressing cell.

This protocol evaluated the following criteria: System Suitability, Specificity, Limit of Detection, Repeatability, and Intermediate Precision.

Testing materials included three lots of CD123-CAR expressing cells, one lot of CS1-CAR expressing cells, untransduced primary T cells, and CD-CHEX-PLUS (stabilized blood manufactured from normal human peripheral blood leukocytes and erythrocytes).

Materials and Equipment

All equipment will be qualified or calibrated prior to use.

| Item/Part Number | Vendor | Model Number |
|---|---|---|
| 2 -8° C. Refrigerator | Thermo Fisher | TSX2320FA |
| MACSQuant | Miltenyi | MACSQuant |
| Analyzer 10 | | Analyzer 10 |
| Centrifuge | Thermo Scientific | 7450 |

Reagents

| Reagent | Vendor | Catalog Number |
|---|---|---|
| Anti idiotype clone 1-E06 | Lake Pharma | Lot number TP26921F |
| | | (1.05 mg/mL) |
| CD3 - PE | BD | 347347 |
| | Biosciences | |
| Streptavidin - APC | BD | 554067 |
| | Biosciences | |
| Anti EGFR - Biotin | R&D | FAB9577B |
| | systems | |
| Anti-Rat Alexa Fluor 647 | Invitrogen | A10540 |
| DAPI | Invitrogen | D21490 |
| CTS ™ OpTmizer ™ T Cell | Gibco | A1048501 |
| Expansion SFM | | |
| CD - CHEX PLUS | Streck | 213325 |
| Flow Cytometry Staining | R&D | FC001 |
| Buffer (1X) | Systems | |
| Human TruStain FcX (Fc | Biolegend | 422302 |
| Receptor Blocking Solution) | | |
| MACS Bleach Solution | Miltenyi | 130-093-663 |
| | Biotec | |
| MACSQuant Calibration | Miltenyi | 130-093-607 |
| Beads | Biotec | |
| MACSQuant Running Buffer | Miltenyi | 130-092-747 |
| | Biotec | |
| MACSQuant Storage Solution | Miltenyi | 130-092-748 |
| | Biotec | |
| MACSQuant Washing | Miltenyi | 130-092-749 |
| Solution | Biotec | |
| Round-Bottom Polystyrene | Falcon | 352052 |
| Tubes 12 × 75 mm | | |
| 96-well plate | Fisher | 168136 |

Procedure

Preparation of T Cells and Cell Product

Required numbers of vials of each cell type were obtained from a −150° C. freezer. The vials were thawed in a 37° C. water bath until only a sliver of ice remained, and the vials were not shaken or stirred while thawing.

The thawed cells were into respective 15 mL centrifuge tubes using a 1000 μL pipette. 10 mL of CTS OpTmizer media was added to each 15 mL tube. The tubes were centrifuged at 200×G for 6 minutes and the supernatant was discarded.

The cell pellet was resuspended in 5 mL CTS OpTmizer media and placed into respective T25 flasks and in a 37° C. incubator for at least 1 hour. After this incubation, the cell suspensions were transferred to respective 15 mL centrifuge tubes and centrifuged at 800×G for 3 minutes. The supernatant was discarded and the cell pellet was resuspended in flow cytometry staining buffer to obtain a density of 5e⁵ cells per 100 μL.

Preparation of CD CHEX PLUS

A vial of CD CHEX PLUS was removed from the refrigerator and warmed to room temperature (18-30° C.) for 15 minutes before use. The vial was then held horizontally between palms of the hands and rolled vial back and forth for 20 to 30 seconds. Gentle inversions (at least 8-10 times) were used to mix the product until all cells are thoroughly suspended. If the vial sits for 30 min on the bench, gently invert the vial 5 times immediately before sampling.

200 μL of the CD Chex Plus reagent was aliquoted per well into a 15 mL tube and the original vial was returned to refrigeration to ensure maximum open vial stability. 3 mL of ACK lyse were added and mixed by pipetting up and down three times. The vial was then incubated for 10±1 minutes at room temperature.

The cells were centrifuged for 3 minutes at 800×G at room temperature, the supernatant was removed, and then the cells were resuspended in 200 μL of Staining Buffer.

Antibody Cocktail Preparations

Cocktail calculations for 1 well are shown.

Anti-idiotype primary antibody cocktail (all assays utilized the antibody comprising a V_{H} of SEQ ID NO: 13 and a V_{L} of SEQ ID NO: 15).

| Antibody against | | Target | Host | |
|---|---|---|---|---|
| (Target Protein) | Conjugate | sp. | sp. | μL per reaction |
| CD3 | PE | Human | Mouse | 5 |
| Anti - idiotype | None | Human | Rat | 1.4 |

| Staining buffer | | 13.6 |
|---|---|---|
| | Total | 25 |

Anti-EGFR Primary Antibody Cocktail

| Antibody against | | Target | Host | |
|---|---|---|---|---|
| (Target Protein) | Conjugate | sp. | sp. | μL per reaction |
| CD3 | PE | Human | Mouse | 5 |
| Anti - EGFR | Biotin | Human | Mouse | 10 |

| Staining buffer | | 35 |
|---|---|---|
| | Total | 50 |

Anti-Rat Alexa Fluor 647 Secondary Cocktail

| Antibody against | | Target | Host | |
|---|---|---|---|---|
| (Target Protein) | Conjugate | sp. | sp. | μL per reaction |
| Rat IgG | Alexa Fluor | Human | Rat | 1 |
| | 647 | | | |

| Staining buffer | | 99 |
|---|---|---|
| | Total | 100 |

Streptavidin APC Secondary Cocktail

| Reagent | Conjugate | Binding Target | μL per reaction |
|---|---|---|---|
| Streptavidin | APC | Biotin | 2 |

| Staining buffer | | 98 |
|---|---|---|
| | Total | 100 |

System Suitability

To test the system suitability, the % of CD3+ cells out of the lymphocytes in a CD Chex Plus sample was determined and compared to the Expected Range provided by the manufacturer.

Specifically, 100 μL of CD CHEX PLUS was added into a well of a 96 well plate. The plate was centrifuged at 800×G for 3 minutes and the supernatant was discarded. The cell pellet was resuspended with 25 μL of corresponding primary antibody cocktail and incubated at room temperature in dark for 25±5 mins.

Next, 150 μL of staining buffer was added to each well, and the plate was again centrifuged at 800×G for 3 mins and the supernatant was discarded. The resulting cell pellet was resuspended in 100 μL of secondary antibody cocktail and incubated at room temp for 20±5 mins.

Next, 100 μL of staining buffer was added to each well and the plate was again centrifuged at 800×G for 3 mins and the supernatant was discarded. The resulting cell pellet was resuspended in 150 μL of staining buffer, then centrifuged again at 800×G for 3 mins (supernatant discarded), and resuspended in 125 μL of staining buffer. DAPU was not added for suitability testing.

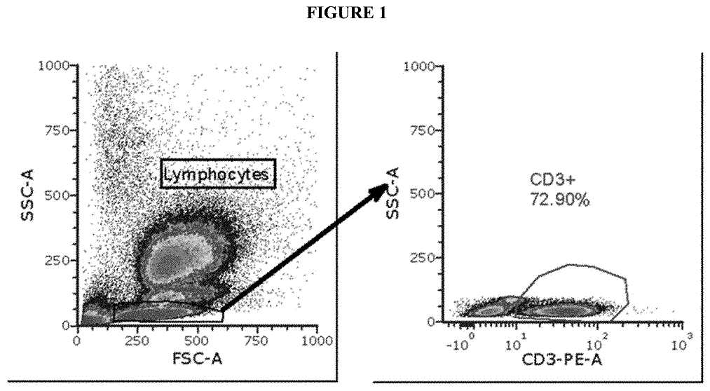

The cells were then analyzed using a MACSQuant 10. Gating of the cells was performed according to the gating strategy in FIG. 1. The % CD3+/Lymphocytes were reported and compared against the manufacturer recommended range for the particular lot used.

Specificity Analysis

The ability to unequivocally detect the CD123 CAR product but not CS1-CAR product by the anti-idiotype reagent was assessed. Specifically, to assess specificity, the anti-idiotype reagent was applied to both CAR products and untransduced T cells. The percentage of cells stained in the CAR cell populations and the untransduced T cells was assessed. Alternately, all three cell products could be stained with an anti-EGFR antibody to detect the EGFR tag that is present on both CAR products.

3×10⁵ cells of each type were added to corresponding wells of a 96 well plate, and the plate was centrifuged at 800×G for 3 minutes. The supernatant was then discarded, and the cell pellet was resuspended with 25 μL of primary antibody cocktail. The resuspended cells were incubate at room temperature in dark for 25±5 mins, and then 150 μL of staining buffer was added to each well. The plate was centrifuged at 800×G for 3 mins, the supernatant, was discarded, and the cell pellet was resuspended in 100 μL of secondary antibody cocktail and incubated at room temp for 20±5 mins. 100 μL of staining buffer was added to each well. The plate was again centrifuged at 800×G for 3 mins, the supernatant was discarded, the cell pellet was resuspended in 150 staining buffer, the plate was centrifuged at 800×G for 3 mins, the supernatant was discarded, the cells were in 125 μL of staining buffer, and then acquired using MACSQuant 10. DAPI was added just before acquisition using the auto reagent add function.

Assessment and Reporting

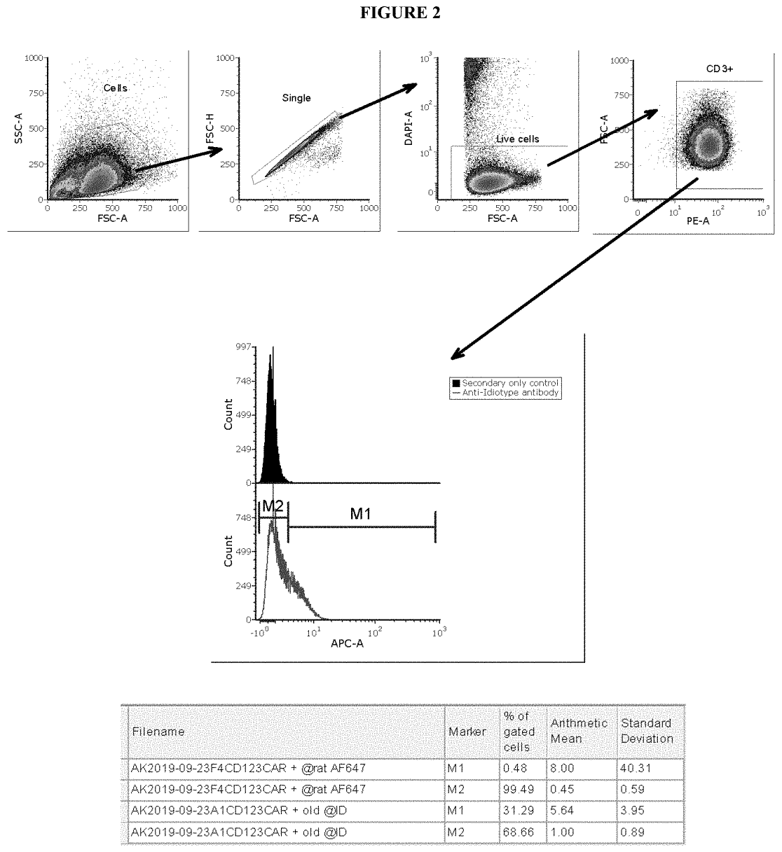

Gating was performed on cells in each well using the gating strategy defined in FIG. 2. The percentage of CAR+ cells expressing the CD123 CAR or the CS1 CAR and untransduced T cells using both the anti-idiotype staining and the anti-EGFR staining was obtained.

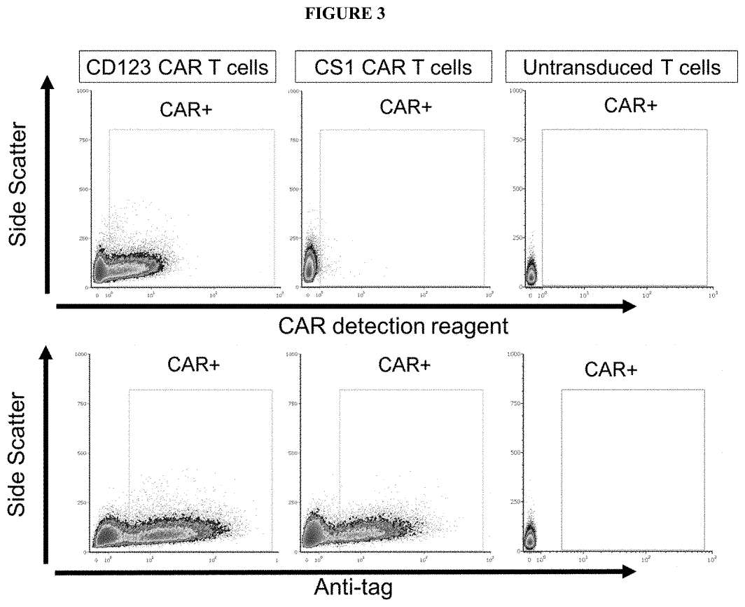

As shown in FIG. 3, the disclosed anti-idiotype antibody is able to specifically detect the CD123-CAR T cells, but not the CS1-CAR T cells.

Limit of Detection (LoD)

The LoD is the lowest concentration of analyte (e.g., CD123-CAR T cells) that can be reliably identified in a given sample and which can be distinguished from a negative control (untransduced T cells). To determine LoD, CD123-CAR T cells were serially diluted into untransduced primary T cells in a 1:2 manner. Each dilution was then assayed using the anti-idiotype cocktail. The 1:2 serial dilution of CAR T cells using untransduced T cells as a diluent was performed until a 1:128 dilution is reached.

3×10⁵ cells of each dilution were to corresponding wells of a 96 well plate, and staining was performed as described above. Gating was performed on cells in each well using the gating strategy defined in FIG. 2 to obtain % of CAR+ cells. The “Limit of Blank (LoB)” was defined as follows:

LoB=mean+2*standard deviations of % of CAR+ cells in Untransduced T cells(wells E9,F9,G9).

The wells with the lowest % CAR+ cells that can be assayed by testing if the Mean of replicate wells>LoB were then determined, and the LoD was defined as follows:

LoB+2*standard deviations of wells with lowest % CAR from 8.5.3.3

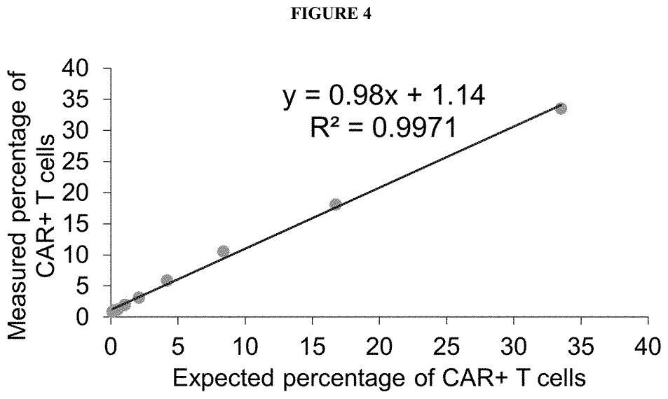

The antibody displayed a highly sensitive response. FIG. 4 shows results of the dilution assessment across a range of 33.5% to 0.1% CAR+ T cells in untransduced T cells, and the results show strong linearity. Over the tested assay range, the assay behaves linearly with a R² value of 0.9971. The sensitivity of detection was 1% CAR+ cells.

Repeatability (Intra-Assay Precision)

The precision of the assay when repeated by the same analyst with the exact same reagents was assessed. To determine repeatability, the assay was repeated three times by the same analyst and % CV of triplicate wells was calculated. Three biological samples were used to assess repeatability.

Three replicates of each of three CD123-CAR T cell samples were added to corresponding wells of a 96 well plate, and staining was performed as described above. The % CV for % CAR were calculated for each sample.

Intermediate Precision (Inter-Assay Precision)

The variation in the assay when repeated by different analysts was also assessed. A second analyst repeated the assay as outlined in the system suitability and the repeatability sections. Cell staining and gating was performed as described above.

### Example 2—Assessment Across Multiple Lots

Five lots of CD123-CAR T cells with a CD19t surrogate tag and four lots of CS1-CAR T cells with a CD19t surrogate tag were manufactured and assessed using the disclosed anti-idiotype antibody comprising a variable heavy chain domain (V_{H}) of SEQ ID NO: 13 and a variable light chain domain (V_{L}) of SEQ ID NO: 15.

As shown in the table below, the antibody was able to detect CD123-CART cells at a level equivalent to surrogate tag staining, but did not detect any of the CS1-CAR T cells lots.

| | | % CAR+ determined | % CAR+ determined |
|---|---|---|---|
| | | by CAR detection | by surrogate tag |
| T cells | Lot # | reagent | staining |
| CD123 - CAR | 1 | 42.0% | 43.7% |
| | 2 | 34.2% | 41.7% |
| | 3 | 34.5% | 47.8% |
| | 4 | 53.6% | 62.2% |
| | 5 | 33.5% | 38.3% |
| CS1 - CAR | 1 | 0.6% | 25.6% |
| | 2 | 1.4% | 31.9% |
| | 3 | 1.2% | 13.2% |
| | 4 | 0.9% | 34.2% |
| Untransduced | N/A | 0.1% | 0.0% |

### Example 3—Assessment in Blood Samples

The ability to assess and detect CD123 using the disclosed anti-idiotype antibodies is not only important from a manufacturing perspective, but also from a clinical perspective. For instance, the disclosed antibodies may be used to periodically assess CAR T cell persistence in clinical trials or during treatment regimen. Accordingly, the ability of the disclosed antibodies to function in blood is clinically and commercially valuable.

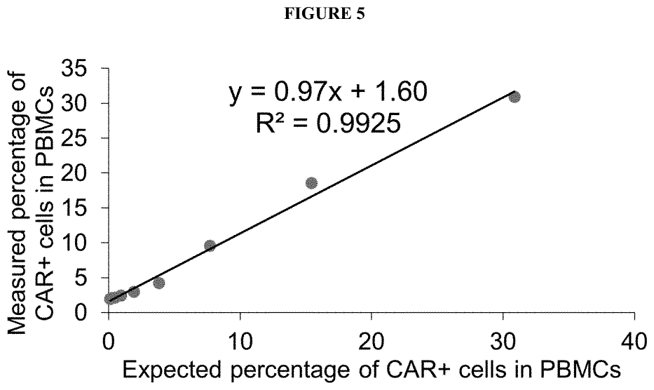

To assess the sensitivity of the disclosed antibodies for clinical applications, CD123-CAR T cells were spiked into whole blood derived PBMCs to generate samples with varied CAR+ proportions over a range of 30% to 0.1%. As shown in FIG. 5, the disclosed antibody (comprising a V_{H} of SEQ ID NO: 13 and a V_{L} of SEQ ID NO: 15) behaves linearly over the tested range, with a R² value of 0.9925, which indicates that the antibody maintains strong sensitivity even in blood samples and suggests that the antibody is robust enough for clinical application.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

Further, one skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

What is claimed:

1. An antibody or antigen-binding fragment comprising a heavy chain variable (V_{H}) region and a light chain variable (V_{L}) region, the V_{H} and V_{L} regions comprising a heavy chain complementarity determining region 1 (CDRH1) comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a light chain complementarity determining region 1 (CDRL1) comprising EDIYX₁X₂ (SEQ ID NO: 10), a CDRL2 comprising X₃AX₄ (SEQ ID NO: 11), and a CDRL3 comprising QQX₅X₆X₇YPX₈T (SEQ ID NO: 12),
wherein X₁ is a polar amino acid; X₂ is selected from the group consisting of serine (S), threonine (T), asparagine (N), glutamine (Q), cysteine (C), glycine (G), and proline (P); X₃ is selected from the group consisting of aspartic acid (D), glutamic acid (E), serine (S), threonine (T), asparagine (N), and glutamine (Q); X₄ is a polar amino acid; X₅ and X₆ are selected from the group consisting of arginine (R), histidine (H), lysine (K), alanine (A), valine (V), isoleucine (I), leucine (L), methionine (M), phenylalanine (F), tyrosine (Y), and tryptophan (W); X₇ is selected from the group consisting of aspartic acid (D), glutamic acid (E), serine (S), threonine (T), asparagine (N), and glutamine (Q); and X₈ is selected from the group consisting of alanine (A), valine (V), isoleucine (I), leucine (L), methionine (M), phenylalanine (F), tyrosine (Y), and tryptophan (W).

2. The antibody or antigen-binding fragment of claim 1, wherein X₁ is serine (S) or asparagine (N); X₂ is asparagine (N) or glycine (G); X₃ is aspartic acid (D) or asparagine (N); wherein X₄ is serine (S) or asparagine (N); X₅ and X₆ are histidine (H) or tyrosine (Y); X₇ is aspartic acid (D) or asparagine (N); or X₈ is leucine (L) or tyrosine (Y).

3. The antibody or antigen-binding fragment of claim 1, wherein X₁ is serine (S) or asparagine (N); X₂ is asparagine (N) or glycine (G); X₃ is aspartic acid (D) or asparagine (N); wherein X₄ is serine (S) or asparagine (N); X₅ and X₆ are histidine (H) or tyrosine (Y); X₇ is aspartic acid (D) or asparagine (N); and X₈ is leucine (L) or tyrosine (Y).

4. The antibody or antigen-binding fragment of claim 1, comprising:
a) a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYSN (SEQ ID NO: 4), a CDRL2 comprising DAS (SEQ ID NO: 5), and a CDRL3 comprising QQHHDYPLT (SEQ ID NO: 6); or
b) a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYNG (SEQ ID NO: 7), a CDRL2 comprising NAN (SEQ ID NO: 8), and a CDRL3 comprising QQYYNYPYT (SEQ ID NO: 9).

5. The antibody or antigen-binding fragment of claim 1, wherein:
a) the V_{H} region comprises
EVQLQQSGAELVRPGASVRLSCTTSGFNIKDSFIHWVKQRTEQGLEWIGRI DPEDDETKYAPKFQGKATITADTSSNTAYLQLSSLTSEDTAVYYCASPIYG SREAWFAYWGQGTLVTVSA (SEQ ID NO: 13) and the V_{L} comprises DIQMTQSPASLSASLGETVTIECLASEDIYSNLAWYQQKPGKSPQLLIYDA SSLQDGVPSRFSGSESGTQYSLEINSLQSEDAATYFCQQHHDYPLTFGSGT KLEIK (SEQ ID NO: 14); or
b) the V_{H} region comprises
EVQLQQSGAELVRPGASVRLSCTTSGFNIKDSFIHWVKQRTEQGLEWIGRI DPEDDETKYAPKFQGKATITADTSSNTAYLQLSSLTSEDTAVYYCASPIYG SREAWFAYWGQGTLVTVSA (SEQ ID NO: 13) and the V_{L} comprises DIQMTQSPASLSASLGETVTIECRASEDIYNGLAWYQQKPGKSPQLLIYNA NSLHTGVPSRFSGSGSGTQYSLKINSLQSEDVASYFCQQYYNYPYTFGAG TKLELK (SEQ ID NO: 15).

6. The antibody or antigen-binding fragment of claim 1, wherein the antibody or antigen-binding fragment specifically binds to an idiotype on an anti-CD123 antibody or an anti-CD123 antigen-binding fragment.

7. The antibody or antigen-binding fragment of claim 6, wherein the anti-CD123 antibody or anti-CD123 antigen-binding fragment comprises the V_{L} domain of
DVQITQSPSYLAASPGETITINCRASKSISKDLAWYQEKPGKTNKLLIYSGSTLQS GIPSRFSGSGSGTDFTLTISSLEPEDFAMYYCQQHNKYPYTFGGGTKLEIK (SEQ ID NO: 20) and/or the V_{H} domain of
QVQLQQPGAELVRPGASVKLSCKASGYTFTSYWMNWVKQRPDQGLEWIGRIDP YDSETHYNQKFKDKAILTVDKSSSTAYMQLSSLTSEDSAVYYCARGNWDDYWG QGTTLTVSS (SEQ ID NO: 21).

8. The antibody or antigen-binding fragment of claim 6, wherein the anti-CD123 antigen-binding fragment is incorporated into a chimeric antigen receptor (CAR).

9. The antibody or antigen-binding fragment of claim 8, wherein the CAR comprises:
a) an IgG hinge or a modified IgG hinge,
b) a transmembrane domain,
c) a co-stimulatory signaling domain, and
d) T cell receptor zeta chain signaling domain.

10. The antibody or antigen-binding fragment of claim 9, wherein the co-stimulatory signaling domain is selected from the group consisting of: a CD27 co-stimulatory signaling domain, a CD28 co-stimulatory signaling domain, a 4-1BB co-stimulatory signaling domain, and an OX40 co-stimulatory signaling domain; and wherein the transmembrane domain comprises a transmembrane portion of CD28, CD4, CD8, 4-1BB, CD27, ICOS, OX40, HVEM, or CD30.

11. The antibody or antigen-binding fragment of claim 8, wherein the CAR comprises a V_{L} domain comprising SEQ ID NO: 20 and V_{H} domain comprising SEQ ID NO: 21, a CD28 transmembrane domain, a co-stimulatory domain comprising SEQ ID NO: 24 or SEQ ID NO: 25, and a CD3ζ domain comprising SEQ ID NO: 48.

12. The antibody or antigen-binding fragment of claim 8, wherein the CAR comprises a V_{L} domain comprising SEQ ID NO: 22 and V_{H} domain comprising SEQ ID NO: 23, a CD28 transmembrane domain, a co-stimulatory domain comprising SEQ ID NO: 24 or SEQ ID NO: 25, and a CD3ζ domain comprising SEQ ID NO: 48.

13. The antibody or antigen-binding fragment of claim 8, wherein the CAR comprises SEQ ID NO: 49 or SEQ ID NO: 50.

14. A nucleotide sequence encoding an antibody or antigen-binding fragment comprising:
(SEQ ID NO: 16)
a) GAGGTTCAGCTGCAGCAGTCTGGGGCAGAGCTTGTGAGGCCAGG
GGCCTCAGTCAGGTTGTCCTGCACAACTTCTGGCTTCAACATTAAAG
ACTCCTTTATTCACTGGGTGAAGCAGAGGACTGAACAGGGCCTGGAG
TGGATTGGAAGGATTGATCCTGAGGATGATGAAACTAAATATGCCCC
GAAATTCCAGGGCAAGGCCACTATAACAGCAGACACATCCTCCAACA
CAGCCTACCTGCAGCTCAGCAGCCTGACATCTGAGGACACTGCCGTC
TATTACTGTGCTAGCCCCATCTACGGTAGTAGAGAGGCCTGGTTTGC
TTACTGGGGCCAAGGGACTCTGGTCACTGTCTCTGCA
and
(SEQ ID NO: 17)
GACATCCAGATGACACAGTCTCCAGCTTCCCTGTCTGCATCTCTGGG
AGAAACTGTCACCATCGAATGTCTAGCAAGTGAAGACATTTACAGTA
ATTTAGCGTGGTATCAGCAGAAGCCAGGGAAATCTCCTCAGCTCCTG
ATCTATGATGCAAGTAGCTTGCAAGATGGGGTCCCATCACGGTTCAG
TGGCAGTGAATCTGGCACACAGTATTCTCTCGAGATCAACAGCCTGC
AATCTGAAGATGCCGCGACTTATTTCTGTCAACAGCATCATGATTAT
CCTCTCACGTTCGGTTCTGGGACCAAGCTGGAGATCAAA;
or
(SEQ ID NO: 18)
b) GAGGTTCAGCTGCAGCAGTCTGGGGCAGAGCTTGTGAGGCCAGG
GGCCTCAGTCAGGTTGTCCTGCACAACTTCTGGCTTCAACATTAAAG
ACTCCTTTATTCACTGGGTGAAGCAGAGGACTGAACAGGGCCTGGAG
TGGATTGGAAGGATTGATCCTGAGGATGATGAAACTAAATATGCCCC
GAAATTCCAGGGCAAGGCCACTATAACAGCAGACACATCCTCCAACA
CAGCCTACCTGCAGCTCAGCAGCCTGACATCTGAGGACACTGCCGTC
TATTACTGTGCTAGCCCCATCTACGGTAGTAGAGAGGCCTGGTTTGC
TTACTGGGGCCAAGGGACTCTGGTCACTGTCTCTGCA
and
(SEQ ID NO: 19)
GACATCCAGATGACACAGTCTCCAGCTTCCCTGTCTGCATCTCTGGG
AGAAACTGTCACCATCGAATGTCGAGCAAGTGAGGACATTTACAATG
GTTTAGCATGGTATCAGCAGAAGCCAGGGAAATCTCCTCAGCTCCTG
ATCTATAATGCAAATAGCTTGCATACTGGGGTCCCATCACGGTTCAG
TGGCAGTGGATCTGGTACACAGTATTCTCTCAAGATAAACAGCCTGC
AGTCTGAAGATGTCGCAAGTTATTTCTGTCAACAGTATTACAATTAT
CCGTACACGTTTGGAGCTGGGACCAAGCTGGAACTGAAA.

15. An expression vector comprising the nucleotide sequence of claim 14.

16. A method of expanding or activating immune cells that express an anti-CD123 chimeric antigen receptor (CD123-CAR) comprising contacting in vitro a population of immune cells that express a CD123-CAR with an anti-idiotype antibody or antigen-binding fragment that specifically binds to an idiotype of the CD123-CAR, wherein the CD123-CAR comprises a scFv that binds to CD123, a hinge domain, a transmembrane domain, a co-stimulatory domain, and T cell receptor zeta chain signaling domain.

17. The method of claim 16, wherein the anti-idiotype antibody or antigen-binding fragment comprises:
a) a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYSN (SEQ ID NO: 4), a CDRL2 comprising DAS (SEQ ID NO: 5), and a CDRL3 comprising QQHHDYPLT (SEQ ID NO: 6); or
b) a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYNG (SEQ ID NO: 7), a CDRL2 comprising NAN (SEQ ID NO: 8), and a CDRL3 comprising QQYYNYPYT (SEQ ID NO: 9).

18. The method of claim 16, wherein the anti-idiotype antibody or antigen-binding fragment comprises:
a) a V_{H} region comprising
EVQLQQSGAELVRPGASVRLSCTTSGFNIKDSFIHWVKQRTEQGLEWIGRI DPEDDETKYAPKFQGKATITADTSSNTAYLQLSSLTSEDTAVYYCASPIYG SREAWFAYWGQGTLVTVSA (SEQ ID NO: 13) and a V_{L} comprising DIQMTQSPASLSASLGETVTIECLASEDIYSNLAWYQQKPGKSPQLLIYDA SSLQDGVPSRFSGSESGTQYSLEINSLQSEDAATYFCQQHHDYPLTFGSGT KLEIK (SEQ ID NO: 14); or
b) a V_{H} region comprising
EVQLQQSGAELVRPGASVRLSCTTSGFNIKDSFIHWVKQRTEQGLEWIGRI DPEDDETKYAPKFQGKATITADTSSNTAYLQLSSLTSEDTAVYYCASPIYG SREAWFAYWGQGTLVTVSA (SEQ ID NO: 13) and a V_{L} comprising DIQMTQSPASLSASLGETVTIECRASEDIYNGLAWYQQKPGKSPQLLIYNA NSLHTGVPSRFSGSGSGTQYSLKINSLQSEDVASYFCQQYYNYPYTFGAG TKLELK (SEQ ID NO: 15).

19. The method of claim 16, wherein the co-stimulatory signaling domain of the CD123-CAR is selected from the group consisting of: a CD27 co-stimulatory signaling domain, a CD28 co-stimulatory signaling domain, a 4-1BB co-stimulatory signaling domain, and an OX40 co-stimulatory signaling domain; and wherein the transmembrane domain comprises a transmembrane portion of CD28, CD4, CD8, 4-1BB, CD27, ICOS, OX40, HVEM, or CD30.

20. The method of claim 16, wherein the scFv of the CD123-CAR comprises SEQ ID NOs: 20 and 21 or SEQ ID NOs: 22 and 23.

21. The method of claim 16, wherein the CD123-CAR comprises SEQ ID NO: 49 or SEQ ID NO: 50.

22. The method of claim 16, wherein the immune cells are T cells or natural killer (NK) cells.

23. A method of detecting the presence of a CD123-CAR in a sample comprising, contacting a sample comprising immune cells that are suspected of expressing a CD123-CAR with an anti-idiotype antibody or antigen-binding fragment that specifically binds to an idiotype of the CD123-CAR and quantifying the number of cells expressing the CD123-CAR, wherein the CD123-CAR comprises a scFv that binds to CD123, a hinge domain, a transmembrane domain, at least one co-stimulatory domain, and T cell receptor zeta chain signaling domain.

24. The method of claim 23, wherein the anti-idiotype antibody or antigen-binding fragment comprises:
a) a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYSN (SEQ ID NO: 4), a CDRL2 comprising DAS (SEQ ID NO: 5), and a CDRL3 comprising QQHHDYPLT (SEQ ID NO: 6); or
b) a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYNG (SEQ ID NO: 7), a CDRL2 comprising NAN (SEQ ID NO: 8), and a CDRL3 comprising QQYYNYPYT (SEQ ID NO: 9).

25. The method of claim 23, wherein the anti-idiotype antibody or antigen-binding fragment comprises:
a) a V_{H} region comprising
EVQLQQSGAELVRPGASVRLSCTTSGFNIKDSFIHWVKQRTEQGLEWIGRI DPEDDETKYAPKFQGKATITADTSSNTAYLQLSSLTSEDTAVYYCASPIYG SREAWFAYWGQGTLVTVSA (SEQ ID NO: 13) and a V_{L} comprising DIQMTQSPASLSASLGETVTIECLASEDIYSNLAWYQQKPGKSPQLLIYDA SSLQDGVPSRFSGSESGTQYSLEINSLQSEDAATYFCQQHHDYPLTFGSGT KLEIK (SEQ ID NO: 14); or
b) a V_{H} region comprising
EVQLQQSGAELVRPGASVRLSCTTSGFNIKDSFIHWVKQRTEQGLEWIGRI DPEDDETKYAPKFQGKATITADTSSNTAYLQLSSLTSEDTAVYYCASPIYG SREAWFAYWGQGTLVTVSA (SEQ ID NO: 13) and a V_{L} comprising DIQMTQSPASLSASLGETVTIECRASEDIYNGLAWYQQKPGKSPQLLIYNA NSLHTGVPSRFSGSGSGTQYSLKINSLQSEDVASYFCQQYYNYPYTFGAG TKLELK (SEQ ID NO: 15).

26. The method of claim 23, wherein the immune cells are T cells or natural killer (NK) cells.

27. The method of claim 23, wherein the CD123-CAR comprises a V_{L} domain comprising SEQ ID NO: 20 and V_{H} domain comprising SEQ ID NO: 21, a CD28 transmembrane domain, a co-stimulatory domain comprising SEQ ID NO: 24 or SEQ ID NO: 25, and a CD3ζ domain comprising SEQ ID NO: 48; or wherein the CD123-CAR comprises a V_{L} domain comprising SEQ ID NO: 22 and V_{H} domain comprising SEQ ID NO: 23, a CD28 transmembrane domain, a co-stimulatory domain comprising SEQ ID NO: 24 or SEQ ID NO: 25, and a CD3ζ domain comprising SEQ ID NO: 48.

28. The method of claim 23, wherein the CD123-CAR comprises SEQ ID NO: 49 or SEQ ID NO: 50.

29. The method of claim 23, wherein the sample is a cell culture medium.

30. The method of claim 23, wherein the sample is a blood sample from a subject that has been treated with the immune cells expressing the CD123-CAR.

31. A method of isolating immune cells that express an CD123-CAR from a sample comprising, contacting a sample comprising immune cells that are suspected of expressing an CD123-CAR with a solid support comprising an anti-idiotype antibody or antigen-binding fragment comprising:
a) a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYSN (SEQ ID NO: 4), a CDRL2 comprising DAS (SEQ ID NO: 5), and a CDRL3 comprising QQHHDYPLT (SEQ ID NO: 6); or
b) a CDRH1 comprising GFNIKDSF (SEQ ID NO: 1), a CDRH2 comprising IDPEDDET (SEQ ID NO: 2), and a CDRH3 comprising ASPIYGSREAWFAY (SEQ ID NO: 3), and a CDRL1 comprising EDIYNG (SEQ ID NO: 7), a CDRL2 comprising NAN (SEQ ID NO: 8), and a CDRL3 comprising QQYYNYPYT (SEQ ID NO: 9);
thereby isolating the immune cells that express the CD123-CAR from the sample, wherein the CD123-CAR comprises a scFv that binds to CD123, a hinge domain, a transmembrane domain, a co-stimulatory domain, and T cell receptor zeta chain signaling domain.

32. The method of claim 31, wherein the sample is a cell culture medium.

33. The method of claim 31, wherein the sample is a blood sample from a subject that has been treated with the immune cells expressing the CD123-CAR.

34. The method of claim 31, wherein the solid support comprising a column or beads to which the anti-idiotype antibody or antigen-binding fragment is linked.

35. The method of claim 31, wherein the CD123-CAR comprises SEQ ID NO: 49 or SEQ ID NO: 50.
